(19) United States
(12) Patent Application Publication
(10) Pub. No.: US 2005/0112598 A1
(43) Pub. Date: May 26, 2005

(54) Microarray-based diagnosis of pediatric hearing impairment-construction of a deafness gene chip

(76) Inventors: John H. Greinwald JR., Loveland, OH (US); Richard J. Wenstrup, Cincinnati, OH (US); Bruce J. Aronow, Cincinnati, OH (US); John P. Pestian, Loveland, OH (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/786,518

(22) Filed: Feb. 24, 2004

Related U.S. Application Data
(63) Continuation-in-part of application No. 10/373,978, filed on Feb. 24, 2003.

(51) Int. Cl. C12Q 1/68; C12M 1/34

(52) U.S. Cl. 435/6; 435/287.2

(57) ABSTRACT

The present invention is related to diagnostic arrays comprising primers for various regions of candidate genes involved in hearing loss, specifically pediatric hearing loss. The invention further is directed to methods for diagnosing a cause or risk factor for hearing loss. In some embodiments, these methods include obtaining a sample from a patient; screening the sample for the presence or absence of alleles of at least 5 loci associated with a risk for hearing loss to obtain a result of the screening; and making a diagnosis based upon the result. The present invention is also directed to the amplification of genetic sequence from multiple or single exons for use in the screening of samples.

### RELATED APPLICATIONS

This application is a continuation-in-part of Non-Provisional application Ser. No. 10/373,978, filed on Feb. 24, 2003. The subject matter of the aforementioned application is hereby incorporated by reference in its entirety.

### FIELD OF THE INVENTION

The present invention relates to methods of diagnosing pediatric hearing impairment with a microarray containing capture nucleotide sequences representing a variety of genes associated with congenital hearing loss in children.

### BACKGROUND OF THE INVENTION

Congenital hearing loss represents one of the most common birth defects in the United States. The prevalence of permanent congenital hearing loss (PCHL) is approximately 1.2 per 1000 live births. The cause of PCHL can be conductive, involving defects in the transmission of vibrations to the inner ear, or sensorineural, involving defects in the detection of sound in the inner ear (cochlear) and/or the transmission of the neural signal to the brain (retrocochlear), or a mixture of both (Sirimanna, K S (2002) Semin Neonatal 6:511-519, which is hereby incorporated by reference in its entirety). Half of all cases of sensorineural hearing loss (SNHL) in children have a genetic origin (Morton, C C (1991) Ann NY Acad Sci 630:16-31, which is hereby incorporated by reference in its entirety).

Hearing loss in infants can go undetected for months after birth. Early detection of hearing disorders is key to avoiding learning difficulties later in a child's life. Researchers have found that early intervention and habilitation of infant hearing loss can alleviate most of the developmental and behavior difficulties found in hearing-impaired children (Sirimanna, K S ibid.). Infants provided with amplification before the age of three months scored at nearly 90% of normal on child development tests given between 3 and 4 years of age (Downs, M P (1995) Int J Ped Otorhinolaryngol 32:257-259, which is hereby incorporated by reference in its entirety). It is apparent that the earlier intervention occurs with hearing-impaired children, the greater the enhancement of the acquisition of speech and language skills. Early intervention has been shown to be much more effective than late measures and thus it is desirable that hearing assessment be completed in the perinatal period. As an illustration, as many as two-thirds of the children born in the state of Ohio with a handicapping hearing impairment are not diagnosed at birth (Ohio Dept of Health Infant Hearing Screening Assessment Program (IHSAP) 1998, which is hereby incorporated by reference in its entirety). Nationally, the average age of children at the time of identification of a handicapping hearing loss is 2.5 years. The consequences of delayed identification of hearing loss and subsequent delayed intervention on a child's communication skills are tremendous. The estimated special educational costs for such late-identified hearing-impaired children ranges from $38,000 to $220,000 per child over the course of a K-12 education. Additional estimates of the costs to society for an individual with late diagnosed hearing impairment approaches $1 million—primarily in special educational costs and lost job productivity.

Unfortunately, the screening procedures for hearing loss in infants can be difficult to perform and evaluate and are usually not conclusive as to the exact cause of hearing loss, its nature or severity. Screening procedures for neonates and infants include typanometry, otoacoustic emissions (OAE), auditory brainstem response (ABR), and the auditory response cradle. Typanometry involves taking physical measurements of the infant's middle ear pressure and can rule out hearing problems due to blockages within the middle ear. During OAE testing, the ability for the ear to return sound vibrations of particular frequencies when presented with an auditory stimulus is measured. The test can indicate the presence of intact hair cells in the cochlea. While these procedures are convenient and provide unambiguous results, they only screen for particular abnormalities that cause deafness and cannot detect other causes. With ABR, the electrical response in the brainstem to an auditory stimulus is detected and measured with electrodes. This procedure can be automated and is sensitive, but gives limited frequency information and can misdiagnose PCHL in infants whose brainstem auditory pathways have not yet fully matured. The auditory cradle detects and measures the response of infants to sound stimuli and can test the integrity of the entire auditory system at one time. But the sensitivity and false positive rates for this device limit its usefulness in the screening of PCHL in younger infants (Watkin, P M (2001) Semin Neonatol 6:501-509, which is hereby incorporated by reference in its entirety; Sirimanna, ibid).

In infants with PCHL, the cause of the hearing loss is sensorineural in nearly 80% of these cases, as opposed to conductive. Among cases of sensineuronal hearing loss, roughly half have a genetic etiology. About half of those cases are due to mutations in one particular gene, Gap Junction Beta 2 (GBJ2), which codes for a gap junction protein known as connexin 26. Over 65 different mutations in GBJ2 that cause hearing loss have been identified. One particular mutation, 35 delG, is by far the most common and is found in most Northern European individuals who have mutations in GBJ2 (ACMG Statement (2002) Genet Med 4:162-171, which is hereby incorporated by reference in its entirety). Mutations in 24 other genes have been discovered that cause hearing loss; it is predicted that the number of genes involved in hereditary hearing loss is over 100. Nearly 70% of these cause non-syndromic types of hearing loss (where the only phenotype is the loss of hearing) (Petit, C et al. (2001) Annu Rev Genet 35:589-646, which is hereby incorporated by reference in its entirety). These genes can have an autosomal recessive, autosomal dominant, or X-linked inheritance pattern or be within the mitochondrial DNA. They may require the presence of other genetic or environmental factors to manifest hearing loss. Two different mutations in a particular gene, both of which cause hearing loss, can have different modes of inheritance: for example, one mutant allele of a particular gene can confer a dominant trait while another allele of the same gene confers a recessive trait (Morton, C C (2002) Hum Mol Gen 11:1229-1240, which is hereby incorporated by reference in its entirety). These facts demonstrate the extreme heterogeneity of genetic hearing loss, along with the common and often indistinguishable phenotypes for these mutations.

Molecular genetic screening techniques have begun to make an impact with the evaluation of children with PCHL. More than ⅔ of all states have programs to systematically screen all newborns for hearing loss, using the techniques outlined above; children who test positive for hearing loss in these physical tests are now routinely screened for the most common mutation of GBJ2, using well-established polymerase chain reaction-based protocols (ACMG Statement, ibid.). However, it is impractical and prohibitively expensive to screen for the many other genes associated with hearing loss using these techniques.

### SUMMARY OF THE INVENTION

The present invention relates to diagnostic arrays to be used in pediatric screening for hearing loss. Thus, embodiments of the present invention include microarrays having multiple probe sequences for nucleic acids related to hearing loss and methods for using such arrays.

One embodiment of the invention is a method for diagnosing a cause or a risk factor for hearing loss, that includes obtaining a sample from a patient, amplifying genetic sequences found in the sample, screening the sample for the presence or absence of alleles associated with a risk for hearing loss and making a diagnosis based upon the result of the screening. Some embodiments feature the amplification of genetic sequences by polymerase chain reaction. Additional embodiments include the amplification of genetic sequences performed using a primer sequence found in Tables 2-10. In some embodiments, the genetic sequences that are amplified are found in genes selected from the group consisting of CDH23, MYO7A, OTOF, SLC26A4, USH2A, KCNQ1, KCNE1, GJB2 and GJB6. Some embodiments feature genetic sequences of at least two adjacent exons. Some of these embodiments contain multiple adjacent exons selected from the group consisting of CDH23 exons 2-3, CDH23 exons 4-6, CDH exons 7-9, CDH23 exons 10-11, CDH23 exons 12-13, CDH23 exons 14-16, CDH23 exons 17-21, CDH23 exons 22-27, CDH23 exons 28-31, CDH23 exons 32-36, CDH23 exons 37-43, CDH23 exons 44-46, CDH23 exons 47-53, CDH23 exons 53-68, GJB2 exons 1-2, GJB6 exons 1-4, KCNE1 exons 1-2, KCNQ1 exons 3-6, KCNQ1 exons 7-10, KCNQ1 exons 12-15, MYO7A exons 5-14, MYO7A exons 16-21, MYO7A exons 16-18, MYO7A exons 22-26, MYO7A exons 28-35, MYO7A exons 36-44, MYO7A exons 45-49, OTOF exons 4-5, OTOF exons 6-8, OTOF exons 9-11, OTOF exons 12-25, OTOF exons 16-25, OTOF exons 16-18, OTOF exons 16-20, OTOF exons 19-20, OTOF exons 21-25, OTOF exons 16-39, OTOF exons 26-39, OTOF exons 40-47, SLC26A4 exons 1-3, SLC26A4 exons 4-6, SLC26A4 exons 11-18, SLC26A4 exons 19-21, USH2A exons 1-3, USH2A exons 5-9, USH2A exons 10-11, USH2A exons 12-13, USH2A exons 15-16 and USH2A exons 17-20. Other embodiments comprise genetic sequences from a single exon. Some of these embodiments contain exon sequences selected from the group consisting of GJB2 exon 2, KCNE1 exon 3, KCNE1 exon 4, KCNQ1 exon 1, KCNQ1 exon 2, KCNQ1 exon 11, KCNQ1 exon 16, MYO7A exon 1, MYO7A exon 2, MYO7A exon 3, MYO7A exon 4, MYO7A exon 15, MYO7A exon 21, MYO7A exon 27, OTOF exon 1, OTOF exon 2, OTOF exon 3, USH2A exon 4, USH2A exon 14 and USH2A exon 21.

Additional embodiments of the invention are methods for diagnosing a cause or a risk factor for hearing loss, that includes obtaining a sample from a patient, screening the sample for the presence or absence of alleles of at least 5 loci associated with a risk for hearing loss wherein said loci comprise sequences found in genes selected from the group consisting of CDH23, MYO7A, OTOF, SLC26A4, USH2A, KCNQ1, KCNE1, GJB2 and GJB6 and making a diagnosis based upon the result of the screening. In some of these additional embodiments, the amount of the genetic material in the sample is augmented before screening. In some of these embodiments, the augmentation is performed by polymerase chain reaction. In some embodiments, the augmentation utilizes a primer selected from Tables 2-10. In other embodiments, screening is conducted directly, without a prior amplification or augmentation. Sequences for screening in some embodiments comprise sequence from at least two adjacent exons. In some of these embodiments, sequence from multiple adjacent exons comprises sequence selected from the group consisting of CDH23 exons 2-3, CDH23 exons 4-6, CDH exons 7-9, CDH23 exons 10-11, CDH23 exons 12-13, CDH23 exons 14-16, CDH23 exons 17-21, CDH23 exons 22-27, CDH23 exons 28-31, CDH23 exons 32-36, CDH23 exons 37-43, CDH23 exons 44-46, CDH23 exons 47-53, CDH23 exons 53-68, GJB2 exons 1-2, GJB6 exons 1-4, KCNE1 exons 1-2, KCNQ1 exons 3-6, KCNQ1 exons 7-10, KCNQ1 exons 12-15, MYO7A exons 5-14, MYO7A exons 16-21, MYO7A exons 16-18, MYO7A exons 22-26, MYO7A exons 28-35, MYO7A exons 36-44, MYO7A exons 45-49, OTOF exons 4-5, OTOF exons 6-8, OTOF exons 9-11, OTOF exons 12-25, OTOF exons 16-25, OTOF exons 16-18, OTOF exons 16-20, OTOF exons 19-20, OTOF exons 21-25, OTOF exons 16-39, OTOF exons 26-39, OTOF exons -47, SLC26A4 exons 1-3, SLC26A4 exons 4-6, SLC26A4 exons 11-18, SLC26A4 exons 19-21, USH2A exons 1-3, USH2A exons 5-9, USH2A exons 10-11, USH2A exons 12-13, USH2A exons 15-16 and USH2A exons 17-20. Sequences for screening in some embodiments comprise sequence from a single exon. In some of these embodiments, sequence from a single exon comprises sequence selected from the group consisting of GJB2 exon 2, KCNE1 exon 3, KCNE1 exon 4, KCNQ1 exon 1, KCNQ1 exon 2, KCNQ1 exon 11, KCNQ1 exon 16, MYO7A exon 1, MYO7A exon 2, MYO7A exon 3, MYO7A exon 4, MYO7A exon 15, MYO7A exon 21, MYO7A exon 27, OTOF exon 1, OTOF exon 2, OTOF exon 3, USH2A exon 4, USH2A exon 14 and USH2A exon 21.

Another embodiment of the invention is a diagnostic hearing loss microarray that includes at least 5 sequences that are indicative of the presence or the absence of an allele associated with a risk for hearing loss, wherein the sequences are selected from the group consisting of CDH23, MYO7A, OTOF, SLC26A4, USH2A, KCNQ1, KCNE1, GJB2 and GJB6. In some embodiments, a microarray of the invention comprises multiple adjacent exons. In some of these embodiments, sequence from multiple adjacent exons comprises sequence selected from the group consisting of CDH23 exons 2-3, CDH23 exons 4-6, CDH exons 7-9, CDH23 exons 10-11, CDH23 exons 12-13, CDH23 exons 14-16, CDH23 exons 17-21, CDH23 exons 22-27, CDH23 exons 28-31, CDH23 exons 32-36, CDH23 exons 37-43, CDH23 exons 44-46, CDH23 exons 47-53, CDH23 exons 53-68, GJB2 exons 1-2, GJB6 exons 1-4, KCNE1 exons 1-2, KCNQ1 exons 3-6, KCNQ1 exons 7-10, KCNQ1 exons 12-15, MYO7A exons 5-14, MYO7A exons 16-21, MYO7A exons 16-18, MYO7A exons 22-26, MYO7A exons 28-35, MYO7A exons 36-44, MYO7A exons 45-49, OTOF exons 4-5, OTOF exons 6-8, OTOF exons 9-11, OTOF exons 12-25, OTOF exons 16-25, OTOF exons 16-18, OTOF exons 16-20, OTOF exons 19-20, OTOF exons 21-25, OTOF exons 16-39, OTOF exons 26-39, OTOF exons 40-47, SLC26A4 exons 1-3, SLC26A4 exons 4-6, SLC26A4 exons 11-18, SLC26A4 exons 19-21, USH2A exons 1-3, USH2A exons 5-9, USH2A exons 10-11, USH2A exons 12-13, USH2A exons 15-16 and USH2A exons 17-20. In some embodiments, a microarray of the invention comprises sequence from a single exon. In some of these embodiments, sequence from a single exon comprises sequence selected from the group consisting of GJB2 exon 2, KCNE1 exon 3, KCNE1 exon 4, KCNQ1 exon 1, KCNQ1 exon 2, KCNQ1 exon 11, KCNQ1 exon 16, MYO7A exon 1, MYO7A exon 2, MYO7A exon 3, MYO7A exon 4, MYO7A exon 15, MYO7A exon 21, MYO7A exon 27, OTOF exon 1, OTOF exon 2, OTOF exon 3, USH2A exon 4, USH2A exon 14 and USH2A exon 21.

An additional embodiment of the invention is a kit for detecting a candidate gene responsible for hearing loss including a diagnostic hearing loss microarray of the invention that has at least 5 sequences that are indicative of the presence or the absence of an allele associated with a risk for hearing loss, along with buffers and components for use with the microarray. A further embodiment of the invention is the kit described above where the microarray includes a solid support, and further has a plurality of capture nucleotide sequences bound to the solid support, where these sequences are representative of regions of candidate genes for hearing loss, and where the support of the kit is adapted to be contacted with a sample from a patient, the sample including target nucleic acid sequences. Additionally, this embodiment includes the contacting of the sample to the support wherein contacting permits hybridization under stringent conditions of a target nucleic acid sequence and a capture nucleotide sequence representative of regions of candidate genes for hearing loss.

### DETAILED DESCRIPTION OF CERTAIN EMBODIMENTS

There exists a need for a speedy, more reliable and more thorough method of screening newborns for hearing loss and the specific genetic causes of that condition, if any are present. Such a method would allow for more precise diagnoses of the hearing dysfunction present in an afflicted infant and would permit for more rapid and appropriate habilitation for the patient.

Hearing impairment is a fairly common congenital defect in children, with about 1 in 1000 affected newborns (Petit, C ibid.). Though it has long been recognized that heredity plays a large role in hearing impairment, the study of the genetic and biochemical causes of hearing loss have only taken off recently. Physiology of the hearing system and the genetic complexity of deafness have hampered the study of hearing loss. For example, there are only a small number of hair cells (˜10,000) in the cochlea, which are responsible for creating neural signals from the mechanical vibrations of sound. This has prevented the biochemical study of the unique proteins of these cells, which requires large amounts of tissue for the extraction and purification of protein samples.

Traditional studies of genetic inheritance were hampered by the substantial genetic heterogeneity and phenotypic conformity of hearing loss. It is now known that genetic hearing loss can be caused by any number of mutations in one or more of hundreds of genes. Many of these mutations result in non-syndromic hearing loss, without any other phenotype besides deafness. Cultural and social factors ensured a high rate of intermarriage of deaf individuals and marriage between the deaf and those from deaf families, creating multigenic lineages for alleles associated with hearing loss. For these reasons, the discernment of discrete inheritable genetic elements contributing to deafness by traditional techniques was very difficult except in highly isolated populations (Morton, C C (2002) Hum Mol Gen 11:1229-1240, which is hereby incorporated by reference in its entirety). Recently, modern molecular biological techniques have accelerated the pace of discovery, with the first identification of a gene linked to non-syndromic hearing loss, GJB2, in 1997 (Zelante, L et al. (1997) Hum Mol Gen 6:1605-1609, which is hereby incorporated by reference in its entirety). Since then, over sixty genetic loci have been identified and dozen of genes implicated in hearing loss (Petit, C ibid.).

Over the last decade, many states have begun to require physiological screening of infants for hearing problems shortly after birth. The importance of these routine screenings is supported by studies showing that early intervention and habilitation of children with hearing loss can greatly improve their language and communication skills later in life (Downs, M P bid). However, some screening protocols only detect cases of hearing loss due to particular causes; others can have unacceptable rates of false positives and negatives. In addition to these problems with detection, the current exam procedures often provide inadequate information as to nature and even severity of the hearing loss in those infants who test positive, information that would be very helpful in the habilitation of the hearing loss. The habilitation of hearing loss involves the amplification of at least part of the sound spectrum usually detected by the human hearing system; the amount and type of amplification must be carefully monitored and adjusted to ensure that the amplification is both adequate and not excessive. Knowing the precise nature of the hearing defect can facilitate estimation of its severity and determination of which frequencies of sound are affected. More available information on regarding an infant patient's particular hearing deficiencies can help with the adjustment of hearing aid devices.

Microarray technology developed within the last decade can address problems with both the research and clinical detection of hereditary hearing loss. Microarrays were developed in the early 1990s to assist with the mapping of the human genome by speeding up the process of genome sequencing. Briefly, a microarray consists of up to thousands of DNA oligonucleotide probes fixed to a solid support in a sequential manner, each probe in a specific location on the solid support. The probes are usually synthesized directly on the substrate support material and are used to interrogate complex RNA or message populations based on the principle of complementary hybridization. A sample of nucleic acid containing a mixture of various sequences can be labeled and allowed to hybridize with the DNA probes of the microarray. After removal of partially hybridized and unhybridized nucleic acids, the presence of nucleic acids with sequences complementary to the sequences of probe DNAs can be detected via their labels. By the positions of the labeling on the array, the identity of the hybridizing nucleic acids can be ascertained. Microarrays thus provide a rapid and accurate means for analyzing nucleic acid samples. They can be used to detect trace amounts of nucleic acids and to distinguish between nucleic acids differing by as little as a single base, in thousands of samples simultaneously. Microarray technology has been used in the laboratory for RNA detection, nucleic acids sequencing projects and for analyzing transcription profiles of cells and tissues (Lichter, P et al. (2000) Semin Hematol 37:348-357; Tusher, V G et al. (2001) Proc Nat Acad Sci 98:5116-5121; Cook, S A and Rosenzweig, A. (2002) Circ Res 91:559-564; each one of which is hereby incorporated by reference in its entirety).

Microarray technology provides a means to test for the genetic causes of current and potential future hearing loss in infants. Typical microarrays provide sets of 16 to 20 oligonucleotide probe pairs of relatively small length (20mers-25mers) that span a selected region of a gene or nucleotide sequence of interest. The probe pairs used in the oligonucleotide array can also include perfect match and mismatch probes that are designed to hybridize to the same RNA or message strand. The perfect match probe contains a known sequence that is fully complementary to the message of interest while the mismatch probe is similar to the perfect match probe with respect to its sequence except that it contains at least one mismatch nucleotide which differs from the perfect match probe. In one embodiment of the invention, the “perfect match” probe refers to a probe containing sequence that is complementary to the predominant genetic sequence found in a population, while the “mismatch probe” can contain the sequence of a particular genetic variant found in that population that varies from the predominant genetic sequence by one or about a few bases. In this way, an array can distinguish between two alleles for a particular gene that differ only by a small number of bases or just one base. During expression analysis, the hybridization efficiency of messages from a sample nucleotide population are assessed with respect to the perfect match and mismatch probes in order to validate and quantify the levels of expression for many messages simultaneously. As each probe detects one particular sequence polymorphism, an array can detect multiple alleles of the same gene as easily as multiple alleles of a plurality of genes. Additional embodiments of the invention include arrays that can detect a specific allele from a genetic locus, arrays that can detect multiple alleles of the same genetic locus and arrays that detect various alleles from a number of different genetic loci, said alleles being associated with a risk of hearing loss.

In some embodiments of the invention, a sample of nucleic acid extracted from a small blood sample is used to carry out the microarray screening procedure. Once a nucleic acid sample is obtained for an individual, it can be manipulated in a number of ways to prepare the sample for analysis on a microarray. For example, messenger RNA can be converted to copy DNA (cDNA) and both cDNA and genomic DNA can be amplified with polymerase chain reaction-based techniques to increase the sensitivity and signal output. Various means for labeling the nucleic acid for detection on the array exist. These means and the preparatory techniques mentioned above are familiar to those of skill in the art.

The advantages of a microarray-based screening are its accuracy, simplicity, efficiency and extreme cost-effectiveness when employed on a population basis. Current protocols allow for screening of only the most common form of hearing loss, DFNB1, by screening for the three most common, distinct deletion mutations in the gene GJB2. Currently GJB2 screening confers a diagnosis of DFNB1 in only about 20-40% of patients (Bradshaw, J K et al. (2002) Assn Res Otolaryngol 25:96-97; Lim, L H Y et al. (2002) Archives of Otolaryngology Head and Neck Surgery, in press; Green, G E, et al. (1999) JAMA 281:2211-2216; each one of which is hereby incorporated by reference in its entirety). Using conventional technology, screening for each specific mutation of all other genes would requires an infinitely complex and expensive mutliplex experiment. For these reasons, the scaling-up of the conventional screening process to cover rare or recently discovered mutations is logistically difficult and prohibitively expensive (Ferraris, A et al. (2002) Hum Mutation 20:312-320, which is hereby incorporated by reference in its entirety).

However, using microarray technology, screening can be done for multiple alleles associated with hearing impairment simultaneously, indeed for any alleles associated with hearing impairment for which sequence data can be obtained for use in oligonucleotide probe synthesis. Application of this novel technology on a national level makes microarray-based screening an exciting tool for hearing specialists by potentially (more than) doubling the detection rate of pathologic mutations by genetic screening of children with hearing loss. Besides raising the effectiveness of detection methods, other advantages of pinpointing the cause of hearing loss early on in the process byscreening for hearing loss using microarray technology can include alleviating the need for expensive time-consuming tests and the need for the sedation required by some patients to complete some tests. Microarrays containing sequences from multiple alleles can contain sequences from multiple exons within those alleles, from multiple exons within those alleles that are adjacent to one another, from a single exon within the allele, from untranslated regions within the allele including introns and 5′ and 3′ untranslated regions, and from any combination of the above. Some microarrays of the invention can feature some alleles with two or more of the sequence combinations above with other alleles containing additional combinations, fewer combinations or a single configuration of genetic sequence as described above. Other microarrays of the invention comprise genetic sequence from single exons of one or more alleles.

Embodiments of this invention include using the technology alongside current physiological testing procedures as an additional screening method for detecting PCHL from genetic causes, as well as future risk for hearing loss from genetic causes. By allowing the screening of multiple alleles from multiple genes simultaneously, microarray technology can permit the identification of patients who have multiple genetic elements that, when combined, increase their risk for hearing loss. For example, individuals who are heterozygous for recessive mutations in either GJB2 or another gene associated with hearing loss, GJB6, usually have normal hearing, but individuals who are heterozygous for recessive mutations in both of those genes simultaneously can suffer from impaired hearing (Rabionet, R E et al. (2002) Trends Mol Med 8:205-212). In one embodiment of the invention, microarray screening readily identifies individuals who are at risk of hearing loss from the combined effects of multiple alleles from different genes. Some of the alleles that can be detected by an array of the invention include alleles located at modifier gene loci. One such locus has been identified in patients with DFNB26 hearing loss, where the presence of one allele suppresses a deafness phenotype usually associated with the presence of another allele at a different locus (Riazuddin, S et al. (2000) Nat Genet 26:431-4). Other alleles detected by an array of the invention can include alleles associated with risk of hearing loss in combination with environmental factors or aging. For example, Johnson et al. have discoved a gene locus in mice that is strongly associated with age-related hearing loss ((2000) Genomics 70:171-180). In some embodiments of the invention, an array identifies sequences of mitochondial DNA that, alone or in combination with environmental factors, other mitochondrial DNA sequences or nuclear genomic DNA sequences, can place an individual at higher risk for hearing loss. For example, the human mitochondrial DNA mutation A1555G predisposes an individual to hearing loss when that individual is exposed to aminoglycoside antibiotics (Guan, M et al. (2001) Hum Mol Gen 10:573-580). Additional embodiments of the invention screen for one or more alleles that can leave an individual vulnerable to hearing loss when exposed or infected with certain pathogens. Nontypeable Haemophilus influenzae is an example of such a pathogen. Heat stable cytoplasmic proteins released when bacterial cells of this species are disrupted can trigger abundant production of mucin in the middle ear, causing chronic otitis media with effusion (COME), the leading cause of conductive hearing loss in the United States. A particular mucin gene, MUC5AC, was found to be highly expressed in middle ear epithelial cells and overexpressed in the middle ears of individuals diagnosed with COME (Wang, B et al. (2002) J Biol Chem 277:949-957). There can be genetic elements in a patient's genome that modify the reaction of the patient to the bacterial proteins that cause the overexpression of mucin. Particular embodiments of the invention can determine if genetic elements of this type are present. Knowledge of such risk factors as these are valuable to medical personnel, who can more aggressively treat bacterial infections in those patients with genetic risk factors for infection-mediated hearing loss than they would usually do. In another embodiment, an array of the invention is used to detect the presence of alleles associated with syndromes that confer risk for a number of disorders, including hearing loss. Usher syndrome, particularly USH3, and Alport's syndrome are two inherited conditions which often are not associated with distemable phenotypes in infants, but lead to disorders of the retina and nephritis, respectively, later on in life, often accompanied by hearing loss. Both USH3 and Alport's have been linked to mutations in one or a few genes and can be readily detected by the invention (Hone, S et al (2001) Semin Neonatal 6:531-541; Longo, I et al (2002) Kidney Int 61:1947-1956). Other embodiments include the screening of adults and future parents for genetic traits associated with hearing loss, as well as testing blastocyst cells from embryos created from in vitro fertilized eggs. In additional embodiments, an array according to the invention can be used to analyze a plethora of genetic elements from one or more patients in order to discover new interactions between genetic elements that affect risk for hearing loss. As more knowledge is gained on the genotype-phenotype correlations in hereditary deafness, this technology can be of great assistance in better defining the prognosis and severity of hereditary hearing loss in children. This knowledge is especially important in newborns diagnosed with hearing loss, due to the difficulty in determining an accurate hearing level with current testing paradigms, by providing prognostic information on the hearing loss at such an early age.

Microarrays are devices that offer the promise of determining the genotypes at every site of interest in human DNA with great efficiency (Lipshutz, R J et al. (1999) Nat Genet 21:20-24, which is hereby incorporated by reference in its entirety). Variation Detection Arrays (VDAs) have been used to such an end with success (Hacia, J G (1999) Nat Genet 21:42-47; Syvänen, A (1999) Hum Mutat 13:1-10; each one of which is hereby incorporated by reference in its entirety). Unfortunately, a small number of false reads have been determined, giving VDAs an accuracy between 99.93-99.99%. Although remarkable, this error rate is problematic for experiments involving large-scale human genetic variation (˜8×10⁻⁴ per site); signals of some mutations with a low rate of frequency are not always detectable against the background noise generated by such an error rate. However, Cutler et al. have reported the use of a new high density VDA with a novel statistical framework for scoring the genotypes called the Adaptive Background genotype Calling Scheme (ABACUS) that allows for greater than 99.9999% accuracy on over 90% of genotype calls (Cutler, D J et al. (2001) Genome Res 11:1913-1925, which is hereby incorporated by reference in its entirety).

Embodiments of the invention include microarrays and diagnostic methods of employing these microarrays for pediatric screening of genes related to hearing loss. Using the methods described herein, genes associated with the early onset of hearing loss can be identified in candidate populations and these results can allow for prognosis and successful rehabilitation to be made within a time critical period of speech and language development of a child.

A microarray-based mutation screening tool of known genes associated with early onset of hearing loss is feasible using new state-of-the-art technology. The rapid and cost effective screening of genetic variations in children with SNHL enables mutations to be identified. This method allows for accurate predictions of hearing loss severity and prognosis and also allows for successful rehabilitation to be made within a time critical period of speech and language development. In addition, this screening tool can enable diagnosis of disorders which include hearing loss. One such example of syndromic hearing loss is Alport's Syndrome, which causes hereditary nephritis or kidney failure early on, while the loss of hearing does not usually present itself until about 5 years of age. The early detection of children with PCHL and children at risk for hearing difficulties due to genetic mutations can greatly enhance the possibilities for successful intervention and habilitation.

Definitions

Unless defined otherwise, technical and scientific terms used herein have the same meaning as commonly understood by one of ordinary skill in the art to which this invention belongs. One skilled in the art will recognize many methods and materials similar or equivalent to those described herein, which can be used in the practice of the present invention. Indeed, the present invention is in no way limited to the methods and materials described. For purposes of the present invention, the following terms are defined below.

“Hearing loss” is defined as a clinically significant, noticeable or detectable loss of hearing ability, in either one or both ears. It can be profound (quietest sound heard in better ear is >95 dB in volume), severe (quietest sounds heard in better ear are 70 to 95 dB), moderate (quietest sounds heard in better ear are 40 to 70 dB) or mild (quietest sounds heard in better ear are 25 to 40 dB). An individual's hearing loss can be steady in its severity or can be progressive. The onset of hearing loss can be at any age. It can be due, for example, to genetic factors, to environmental factors, to infectious agents, any number of physical injuries, or any combination of the foregoing.

A “label” is any moiety which can be attached to a polynucleotide and provide a detectable signal, and any labels and labeling methods known in the art are applicable for the present invention. For example, the nucleotides (capture and target) can be coupled directly or indirectly with chemical groups that provide a signal for detection, such as chemiluminescent molecules, or enzymes which catalyze the production of chemiluminescent molecules, or fluorescent molecules like fluorescein or cy5, or a time resolved fluorescent molecule like one of the chelated lanthanide metals, or a radioactive compound. Alternatively, the targets can be labeled after they have reacted with the probe by one or more target-specific reporters.

The terms “polynucleotide” and “oligonucleotide” are used in some contexts interchangeably and mean single-stranded and double-stranded polymers of nucleotide monomers, including 2′-deoxyribonucleotides (DNA) and ribonucleotides (RNA). A polynucleotide can be composed entirely of deoxyribonucleotides, entirely of ribonucleotides, or chimeric mixtures thereof. Likewise polynucleotides can be composed of, for example, internucleotide, nucleobase and sugar analogs, including unnatural bases, sugars, L-DNA and modified internucleotide linkages. The capture nucleotide sequence(s) of the invention fall within this scope and the term “primer(s)” is used interchangeably with capture nucleotide sequence(s). “Target nucleotide sequence” refers to a specific candidate gene, the presence or absence of which is to be detected, and that is capable of interacting with a capture nucleotide sequence.

The term “capture” generally refers to the specific association of two or more molecules, objects or substances which have affinity for each other. In specific embodiments of the present invention, “capture” refers to a nucleotide sequence which is present for its ability to associate with another nucleotide sequence, typically from a sample, in order to detect or assay for the sample nucleotide sequence.

Typically, the capture nucleotide sequence has sufficient complementarity to a target nucleotide sequence to enable it to hybridize under selected stringent hybridization conditions, and the Tₘ is generally about 10° to 20° C. above room temperature (e.g., about 37° C). In general, a capture nucleotide sequence can range from about 8 to about 50 nucleotides in length, preferably about 15, 20, 25 or 30 nucleotides. As used herein, “high stringent hybridization conditions” means any conditions in which hybridization will occur when there is at least 95%, preferably about 97 to 100%, nucleotide complementarity (identity) between the nucleic acids. In some embodiments, modifications can be made in the hybridization conditions in order to provide for less complementarity, e.g., about 90%, 85%, 75%, 50%, etc. Among the hybridization reaction parameters which can be varied are salt concentration, buffer, pH, temperature, time of incubation, amount and type of denaturant such as formamide, etc. (See, e.g., Sambrook et al. (1989) Molecular Cloning: A Laboratory Manual (2d ed.) Vols. 1-3, Cold Spring Harbor Press, New York; Hames et al. (1985) Nucleic Acid Hybridization IL Press; Davis et al. (1986) Basic Methods in Molecular Biology, Elsevier Sciences Publishing, Inc., New York; each one of which is hereby incorporated by reference in its entirety.) For example, nucleic acid (e.g., linker oligonucleotides) can be added to a test region (e.g., a well of a multiwell plate—in a preferred embodiment, a 96 or 384 or greater well plate), in a volume ranging from about 0.1 to about 100 or more μl (in a preferred embodiment, about 1 to about 50 μl, most preferably about 40 μl), at a concentration ranging from about 0.01 to about 5 μM (in a preferred embodiment, about 0.1 μM), in a buffer such as, for example, 6×SSPE-T (0.9 M NaCl, 60 mM NaH₂ PO₄, 6 mM EDTA and 0.05% Triton X-100), and hybridized to a binding partner (e.g., a capture nucleotide sequence on the surface) for between about 10 minutes and about at least 3 hours (in a preferred embodiment, at least about 15 minutes) at a temperature ranging from about 4° C. to about 37° C. (in a preferred embodiment, at about room temperature).

The verb “bind” and its conjugated forms, “binding” and “bound,” refer to the physical association of a molecule or physical object or substance with another molecule, object or substance. The binding of one molecule, object or substance to another can be irreversible or reversible and can involve specific portions or regions of the molecules, objects or substances. The binding can be achieved through covalent bonding, through ionic bonding or through the affinity binding of certain molecules, said molecules being inherently part of the molecules, objects or substances being bound or having been bound themselves to molecules, objects or substances before said molecules, objects or substances were bound.

The term “solid support” refers to any solid phase material upon which a capture nucleotide sequence can be attached or immobilized. For example, a solid support can include glass, metal, silicon, germanium, GaAs, plastic, or the like. Solid support encompasses terms such as “resin,” “solid phase,” and “support.” A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled-pore-glass (CPG), or reverse-phase silica. The configuration of a solid support can be in the form of beads, spheres, particles, granules, a gel, a fiber or a surface. Surfaces can be planar, substantially planar, or non-planar. Solid supports can be porous or non-porous, and can have swelling or non-swelling characteristics. A solid support can be configured in the form of a well, depression or other container, slide, plate, vessel, feature or location. A plurality of solid supports can be configured in an array.

“Array” or “microarray” means a predetermined spatial arrangement of capture nucleotide sequences present on a surface of a solid support. The capture nucleotide sequences can be directly attached to the surface, or can be attached to a solid support that is associated with the surface. The array can include one or more “addressable locations,” that is, physical locations that include a known capture nucleotide sequence.

An array can include any number of addressable locations, e.g., 1 to about 100, 100 to about 1000, or 1000 or more. In addition, the density of the addressable locations on the array can be varied. For example, the density of the addressable locations on a surface can be increased to reduce the necessary surface size. Typically, the array format is a geometrically regular shape, which can facilitate, for example, fabrication, handling, stacking, reagent and sample introduction, detection, and storage. The array can be configured in a row and column format, with regular spacing between each location. Alternatively, the locations can be arranged in groups, randomly, or in any other pattern. In one embodiment an array includes a plurality of addressable locations configured so that each location is spatially addressable for high-throughput handling. Examples of arrays that can be used in the invention have been described in, for example, U.S. Pat. No. 5,837,832, which is hereby incorporated by reference in its entirety.

In a two-dimensional array the addressable location is determined by location on the surface. However, in one embodiment the array includes a number of particles, such as beads, in solution. Each particle includes a specific type or types of capture nucleotide sequence(s). In this case the identity of the capture nucleotide sequence(s) can be determined by the characteristics of the particle. For example, the particle can have an identifying characteristic, such as shape, pattern, chromophore, or fluorophore.

“Surface” when used herein refers to the underlying core material of the arrays of the invention. Typically the surface is a solid support and has a rigid or semi-rigid surface. In one embodiment the surface of the support is flat. In other embodiments the surface can include physical features, such as wells, trenches and raised, shaped, or sunken regions. The capture nucleotide sequences that form the array can be attached directly to the surface, or can be attached to a solid support that is itself associated with, such as attached to or contained by, the surface.

Capture nucleotide sequences can be synthesized by conventional technology, e.g., with a commercial oligonucleotide synthesizer and/or by ligating together subfragments that have been so synthesized. For example, preformed capture nucleotide sequences, can be situated on or within the surface of a test region by any of a variety of conventional techniques, including photolithographic or silkscreen chemical attachment, disposition by ink jet technology, electrochemical patterning using electrode arrays, or denaturation followed by baking or UV-irradiating onto filters (see, e.g., Rava et al. (1996) U.S. Pat. No. 5,545,531; Fodor et al. (1996) U.S. Pat. No. 5,510,270; Zanzucchi et al. (1997) U.S. Pat. No. 5,643,738; Brennan (1995) U.S. Pat. No. 5,474,796; PCT WO 92/10092; PCT WO 90115070; each one of which is hereby incorporated by reference in its entirety).

Depending upon the array used in the present invention, the methods of detecting hybridization between a capture nucleotide sequence and a target nucleic acid sequence can vary. For example, target nucleotide sequences can be labeled before application to the microarray. Through hybridization of the target sequence to the capture probe of complementary sequence on the array, the label is bound to the array at a specific location, revealing its identity. Utilization of glass substrates for microarray design has permitted the use of fluorescent labels for tagging target sequences. Fluorescent labels are particularly useful in microarray designs that utilize glass beads as a solid support for the array; these beads can be interrogated using fiber optics and the measurement of the presence and strength of a signal can be automated (Ferguson, J A et al. (1996) Nat Biotechnol 14:1681-1684, which is hereby incorporated by reference in its entirety). Labeling of target DNA with biotin and detection of the hybridized target on the array with antibodies to biotin has also been done (Cutler, D J ibid.).

An “allele” is defined in some embodiments as a sequence or a member of a pair or series of genes or sequences that occupy a specific position, or locus, on a specific chromosome or segment of nucleic acid found within a cell. The term commonly refers to any number of possible nucleotide sequences containing mutations that occur within a particular gene within the genome of an organism. An allele can contain, in comparison to the sequence of the same genetic locus from another chromosome of the same number, any type of mutation or sequence difference, including a deletion mutation, an insertion mutation, a transitional mutation, a duplication or inversion mutation, or any combination of the above mutations. In some embodiments, an “allele” can refer to a particular variant of mitochondrial DNA or nucleic acid sequence derived from mitochondrial DNA.

“Candidate” refers to a genetic sequence, an allele or a gene, or any part of an allele or gene, which is or can be associated with risk, potential, presence or absence of hearing loss. Many suitable candidate sequences, genes and alleles are known in the art and are reported in the literature. Such can be labeled with terms to specify a particular mutation. In other embodiments, candidate sequences contain within themselves particular and discrete mutations, some of which may have been identified, characterized or described in scientific or medical literature. Embodiments of the invention contemplate use of any appropriate candidate sequences, genes, alleles, and mutations associated with hearing loss. A candidate sequence, gene, allele or mutation that is associated with hearing loss can be a sequence whose presence confers a phenotype of hearing loss or a sequence whose presence alters the risk of hearing loss is either a positive or negative manner. As used herein, an “allele that is associated with a risk of hearing loss” can be an allele which reduces or increases the likelyhood of an individual having or developing hearing loss. It can also be an allele which confers a phenotype of hearing loss.

The term “sample,” as used herein, is defined as an amount of biological material which is obtained directly or indirectly from an individual. The biological material can be a fluid including, for example, amniotic fluid, an amount of blood or some portion of a blood sample; it can also be a sample of tissue, cells, waste, lymph, mucus, vaginal discharge, or the like. The sample can be an amount of biological material in its original state as it was upon being obtained from the source individual or the biological source it originated from, or it can be processed, prepared or otherwise manipulated before being brought to the assay processes, methods, techniques or kits described herein.

When defining the source of a sample, for example, a sample from a child or a sample from a fetus, the sample in question can be directly or indirectly obtained from said child or said fetus. A sample can be taken directly from an individual for the expressed purposes of analysis as set forth in embodiments of the present invention or it can be obtained from a source of biological material taken from an individual or isolated from a sample taken from an individual at another time. A sample can be a subset of biological material isolated from another sample.

In some particular embodiments, a “blood sample” refers to a sample of blood obtained from an individual for whom a diagnosis is sought, or some component or derivative of that sample. In other embodiments, “blood sample” can refer to cells contained in the blood that are not originating from the individual from whom the sample of blood was taken. These embodiments can include a sample having blood cells originating from a fetus that can be isolated from a blood sample taken from the individual carrying said fetus, either during or after pregnancy.

The term “epithelial” generally relates to the epithelium, which is membranous tissue composed of one or more layers of cells. These cells form the cover of most internal and external surfaces of the body and its organs. In some embodiments of the present invention, a sample of epithelial cells can be collected from any number of locations on or within the body or an individual or from tissue or fluid samples which were already collected from an individual.

As used herein, “conductive” is commonly used to denote hearing loss due to problems or issues with the external or middle ear. “Sensineuronal” commonly refers to hearing loss due to problems or issues in any location from the inner ear to the cortical hearing centers of the brain. “Syndromic” refers to hearing loss whose appearance or presence is part of a group or pattern of associated characteristics or phenotypes, wherein the hearing loss can be congenital or can appear later in the life of an individual; can be due to genetic factors, to environmental factors or a combination of factors; and can be sensorineuronal, conductive or be a mixture of factors including sensorineuronal factors, conductive factors or both sensorineuronal and conductive factors. “Non-syndromic” refers to hearing loss which is manifested without a group or pattern of associated characteristics or phenotypes.

The term “genetic,” as used herein in association with hearing loss, commonly refers to risk factors or phenotypes of hearing loss or potential hearing loss that are inheritable. Genetic factors in this context include genomic sequences, chromosomal sequences and extra-nuclear nucleic acid sequences including mitochondrial sequences. The manifestation of the genetic elements and factors can be as DNA sequences, as RNA sequences, as aspects of the proteasome on a molecular or visually detectable level or as some other measurable or detectable physical or behavioral trait.

“Environmental” is commonly used to denote those factors or influences that are not explicitly genetic. In some embodiments, environmental factors can include in utero factors present during an individual's gestation period. Other environmental factors can include physical forces, disease agents, nutritional components or chemical compounds to which an individual is exposed or to which the female carrying said individual as an embryo or fetus is exposed.

The term “amplification” refers to the manipulation of the genetic material in a sample that results in a greater amount of genetic material to be present than before the manipulation. In some embodiments of the present invention, amplification takes place before screening steps of the invention and in some embodiments, this amplification is performed through the use of polymerase chain reaction based techniques. Additional embodiments relate to the use of other amplification techniques, which can include modification of the genetic material in addition to the creation of a greater amount of genetic material.

“Exons” refers to genetic sequences containing information that usually directs the assembly of amino acids into polypeptides. Under certain circumstances, it is possible that exon sequences may not direct the assembly or be able to direct the assembly of amino acids into polypeptides. One such circumstance is the presence of one or more mutations upstream from the exon sequences that distrupt the ability of the sequences to direct or be able to direct the synthesis of polypeptides. “Introns” refer to sequences normally found interspersed among exon sequences that do not contain information regarding the order of amino acids found on a polypeptide. It would be understood by a person with skill in the art that the information content of exon sequences may not be limited to the sequence of amino acids in a polypeptide and that genes can contain sequences that are neither exons nor introns. Some examples of genetic sequences that are neither introns nor exons include untranslated regions found before start codons and after stop codons, including sequences that direct the activities of enzymes that are involved in transcription, translation, RNA processing, RNA degradation, the maintenance and replication of chromosomes or other nuclear or cytosolic processes.

Exons that are “adjacent” to one another are found sequentially next to one another in a polypeptide. There may be additional sequence separating exons that are adjacent. An example of such an interluding sequence is an intron. Other sequences may also intervene between two adjacent exons, including spacer regions and any form of untranslated genetic sequence. Sequence from a single exon may be entirely from within the defined boundaries of a particular exon from a particular gene. It may also including other non-exon sequences, such as sequences from one or more introns or other untranslated sequences.

### EXAMPLES

The following examples disclose various applications of the present invention and are not intended to be limiting. These examples can be used in conjunction with conventional pediatric screening methods or as a primary screening tool.

Candidate Genes

### Example 1

### Selection of Candidate Genes

Candidate genes contemplated in the array of the present invention are selected from a variety of sources, to include those derived from literature reviews and those disclosed, for example, in various databases (i.e., NCBI, Celera, Hereditary Hearing Loss Homepage, GeneDis). While a number of candidate genes are known in the art, there still remain candidate genes yet to be discovered and these genes are contemplated within the scope of the present invention based upon their place within the selection criteria. These candidate genes can be prioritized based whether the gene mutation codes for a nonsyndromic or syndromic type phenotype and whether it has a relatively high, medium or low prevalence. The prevalence categories can be based upon the number of families identified with mutations causing hearing loss (high>20 families; medium from 10 to 19 families; low<10 families). Criteria for prioritizing candidate genes for inclusion can be, for example, (in order of descending priority):
1) nonsyndromic-high prevalence;
2) syndromic (but not readily apparent in early childhood);
3) non-syndromic-medium prevalence; and
4) non-syndromic-low prevalence.

These candidate genes can be selected for inclusion based upon:
1) the identification of unambiguous mutations associated with HI; and
2) the association of mutations of the candidate gene with early onset, handicapping HI (<2 yrs of age) and concomitant communications skills delays.

Data are collected on the auditory phenotype, inheritance, and number of exons and base pairs of coding DNA, prevalence and epidemiology of affected pedigrees. The combination of this information enables candidate genes to be selected for inclusion on an array.

The following table is a non-limiting example of candidate genes for inclusion in the array of the present invention:

TABLE 1
Candidate genes causing congenital hearing loss

| | Contig # | | | | coding | | | |
|---|---|---|---|---|---|---|---|---|
| | (start) | | | | cDNA | # of | | # of |
| Genes | Locus Link # | Phenotype | Inheritance | Exons | (bp) | families | Ethnicity/Country | mutants |
| GJB2 | NT009799 | DFNB1 | AR | 2 (4) | 800 | >20 | C > O >> AA | 50+ AR/6 |
| | (1741608) | | | | | (˜30%) | | AD |
| | 2706 | DFNA3 | AD | | | 2 | | |
| | | PPK | | | | <10 | | |
| GJB6 | NT009799 | DFNB1 | AR | 3 (4) | 786 | ?>20 | Spain, Israel | |
| | (1776107) | | | | | | | |
| | 10804 | DFNA3 | AD | | | 1 | | |
| | | Clouston's | AD | | | rare | | |
| SLC26 | NT007933 | DFNB4/EVA/ | AR | 21 | 2300 | >20 | C, O | 50+ |
| A4 | | PDS | | | | (˜5%) | | |
| | 5172 | | | | | | | |
| OTOF | NT005204 | DFNB9 | AR | 48 | 3700 | 6 | India, Lebanon, Israel | 3 |
| | 9381 | AN | AR | | | 1 | US | 2+ |
| MYO7A | NT033927 | DFNB2 | AR | 49 | 6645 | 2 | Tunisian, Chinese | 4 |
| | 4647 | DFNA11 | AD | | | 1 | Japanese | 1 |
| | | USH1B | AR | | | | Diverse | |
| CDH23 | NT024037 | DFNB12 | AR | 68 | 10062 | 5 | Diverse | 7 |
| | 64072 | USH1D | AR | | | 8 | US, Cuban | 7 |
| USH2A | NT004612 | USH2A | AR | 21 | 4700 | 5 | N. European | 10+ |
| | 7399 | | | | | | | |
| KCNQ1 | NT009368 | JLN | AR | 16 | 1750 | >20 | European, but diverse | 30+ |
| | 3784 | | | | | | | |
| KCNE1 | NT011512 | JLN | AR | 3 | 290 | ?10-20 | same | |
| | 3753 | | | | | | | |
| PAX3 | NT005403 | WS1 | AD | 3 (5) | 618 | many | diverse | many |
| | 5077 | | | | | | | |
| Total | | | | 239 | 31660 | | | |
| | | | | | bp | | | |
| | | | | (˜170) | 2300 | for splice | | |
| PCDH15 | | USH1F | AR | 33 | 5900 | 4 | Pakistan, ME | |
| GJA1 | | | AR | 1 | 700 | 14 | ?AA | |
| TECTA | | DFNB21 | AR | 23 | 6450 | 1 | Lebanese | |
| TMIE | | DFNB6 | AR | | 468 | 5 | India, Pakistan, ? | 2 |
| | | ?USH2B? | | | | | | |
| Harmonin | | USH1C | AR | 21 | 4700 | 8 | Acadia, Lebanon | |
| | | ?DFNB18 | AR | | | | | |
| Total | | | | 78 | 18218 | | | |
| | | | | (˜60) | ˜1000 | for splice | | |
| Prestin | | DFNB | AR | 20 | 6696 | 2 | C | |
| TMPRSS3 | | DFNB8/10 | AR | 13 | 1362 | 2 | ME, Pakistan | |
| OTOA | | DFNB22 | AR | 9 | 3264 | 1 | Pakistan | |
| STRC | | DFNB16 | AR | 29 | 5427 | 4 | Pakistan, ME, France | |
| MITF | | WS2 | AD | 8 | 1257 | several | diverse | |
| MYO15 | | DFNB3 | AR | 50+ | 7200+ | 6 | Bali, India | 2 |
| TMC1 | | DFNB7/11 | AR | 20 | | 11 | Pakistan, India | |
| | | DFNA36 | AD | | | 1 | ″ | |
| CLD14 | | DFNB29 | AR | 3 | 720 | 2 | Pakistan | 6 |
| USH3 | | USH3 | AR? | 4 | 360 | 3 | Finnish, Italian | |
| COL4A5 | | Alport | X-Linked | 51 | 5000+ | >20 | diverse | 8 |
| COL4A3 | | ″ | AR | 51 | 5000+ | <10 | diverse | 6 |
| COL4A4 | | ″ | AR | 43 | 5000+ | <10 | diverse | 6 |

Legend:
AN—auditory neuropathy;
??13 unknown;
AR—autosomal recessive;
AD autosomal dominant;
C—Caucasian;
O—Oriental;
AA—African-American;
ME—Middle Eastern

### Example 2

### Production of Representative Capture Oligonucleotides of Candidate Genes

All gene sequences and cDNA structures of the candidate genes are ascertained from resources such as academic and patent literature and analysis of available databases (i.e., NCBI, Celera, Hereditary Hearing Loss Homepage, GeneDis). As with known candidate genes, the gene sequences and cDNA structures of additional genes found to be candidate genes can be determined by known methods in the art. This applies to any mutations of these candidate genes. This detailed analysis of the gene structure is used in the construction of the PCR primers for amplification of coding regions, splicing junctions, identifiable promoters and other indicative regions of the candidate genes.

For example, exon-intron boundaries can be identified for genes from cDNA and genomic sequences using software available in the art such as the large gap tool Sequencher 4.05 (Genecodes, Ann Arbor, Mich.). These cDNA and/or genomic sequences can be derived from, for example, public databases, literature reviews as well as through experimentation. PCR primers are constructed and optimized conditions to PCR amplify these coding sequences are determined in order to produce representative oligonucleotides of the coding sequences of the candidate genes.

One such method of amplifying the coding region of each exon, the splice-site and an approximately 100 bp of each intron is as follows:

Primers can be positioned in the introns. PrimerSelect (DNASTAR) primer algorithm can be utilized to maximize primer design. PCR is performed with 40 ng of genomic DNA in a 12 μl reaction mixture containing 1.50 μl buffer (100 mM TRIS-HCl pH 8.8, 500 mM KCl, 15 mM MgCl₂, 0.01% w/v gelatin); 10 μM each of dCTP, dGTP, dTTP and dATP supplemented with; 2.5 pmol of forward and reverse primers and 0.25 U Taq polymerase. Thirty cycles of amplification is performed at 94° C. for 30s, 55° C. (or optimized temperature) for 30s, 72° C. for 30s, followed by a 10 min extension at 72° C. Reaction products can be resolved on agarose gels, cleaned directly or gel purified (Qiagen Inc., Valencia, Calif.) and confirmed with sequencing.

Primers can also be chosen to amplify only genetic sequence from exons, introns or any other untranslated region of a gene. For example, the following tables contain sequence and amplification product information for primer pairs that have been used to amplify relevant sequences from particular genes implicated in hearing loss: CDH23, KCNE1, KCNQ1, MYO7A, OTOF, SLC26A4 AND USH2A. The amplification products generated by these reactions can be used with the present invention and may contain sequences from one or more exons, introns and other untranslated regions. The particular primers designed represent segments of genetic sequence that have been selected and tested for optimal priming capacity in polymerase chain reaction-type amplification reaction. The primer sequences can have utility in additional procedures, including other augmenting and amplifying procedures, that may be used with the invention. While these exemplary primer sequences possess characteristics that confer usefulness in amplification reactions, a person with skill in the art would understand that these lists of primer sequences are not exclusive for the goal of sequence amplification and that other primer sequences may exist that can be used with the techniques of the invention, including primer sequences for the amplification of hearing loss genes other than those for which primer sequences are listed below.

TABLE 2
CDH23 primers

| | | | | | |
|---|---|---|---|---|---|
| primer name | primer sequence | bases | product size | primer pair | exons |

| | | | | | |
|---|---|---|---|---|---|
| CDH23-1F | CACTGTGCTATACCCAGGATAGGACAATGTTA | 32 | 6886 bp | 1F/1R | 2 to 3 |
| CDH23-1R | TCAGGTGGAAGATGACCTCAACCTGTAAGATC | 32 | | | |
| CDH23-2F | GATACCATCATGACACACTGTGACAAGT | 28 | 1426 bp | 2F/2R | 4 to 6 |
| CDH23-2R | GACTCTTCACCTACACCATGGTGGTCTG | 28 | | | |
| CDH23-3F | TATGTATTCACACACTAACCCTGTGAGATATG | 33 | 12362 bp | 3F/3R | 7 to 9 |
| CDH23-3R | TAGCCCTCAGAGCCTGAGATGCCTACTGGCTC | 32 | | | |
| CDH23-4F | TGAGTCTTTAATGCCCAGAGAGGAG | 25 | 2604 bp | 4F/4R | 10 to 11 |
| CDH23-4R | TGAGATGGAGTCTTACTCTTGTTGC | 25 | | | |
| CDH23-5F | CCAGAAGCTATGGCCGATCAGAGG | 24 | 3425 bp | 5F/5R | 12 to 13 |
| CDH23-5R | GCAACCAAGAGTACTGACAGATACA | 25 | | | |
| CDH23-6F1 | TGTAGGTAGAAGGCGTGCAGGAGCCAGCAGTCGC | 34 | 6878 bp | 6F/6R | 14 to 16 |
| CDH23-6R1 | GGTTCGAGTGTTTGCTGCTCAGCCTTCCGAGTAT | 34 | | | |
| CDH23-6F1b | CCAAAGGAGACGTGCGAGAGGAACAT | 26 | 4601 bp | 6F1b/6R1b | 14 to 16 |
| CDH23-6R1b | TTCCTGAGTAGCCCAGAGTGTCAGG | 25 | | | |
| CDH23-7F | ACCTCAGTCGAGATGTTGAGGCTCCAGGTGTTC | 33 | 13282 bp | 7F/7R | 17 to 21 |
| CDH23-7R | CTATTGCAAGAGCCAGCTCAGAGGGACACAGA | 32 | | | |
| CDH23-8F | GAGGGTTTGATGAGGAGGAACCCAGTCTCCAA | 32 | 12314 bp | 8F/8R | 22 to 27 |
| CDH23-8R | ATTAACTCGCTGGCTCTAGGA1TVCAGTAAGAG | 33 | | | |
| CDH23-9F | GTAGGATGCGTGAAGGGAAGGAAAGGAACT | 30 | 8499 bp | 9F/9R | 28 to 31 |
| CDH23-9R | GTGCACACAGAAGGAGCTCAACCAATGTrGG | 31 | | | |
| CDH23-10F | GTTATGCCGGACAGAGGAAGTGACATGGAGGT | 32 | 7903 bp | 10F/10R | 32 to 36 |
| CDH23-10R | CAAGGATTCGCCTGCTGTGTGGAATTCCATTC | 32 | | | |
| CDH23-11F | GAGTCACATGGAGTGAGTTCAGCCCAGGAGAA | 32 | 11691 bp | 11F/11R | 37 to 43 |
| CDH23-11R | ACAATGACCACGACTGTCTCTTCCAACCAGAC | 32 | | | |
| CDH23-12F3a | TTATGACTTGCTTCTGATCTTCCTTTCTGATG | 32 | 7912 bp | 12F3a/12R3a | 44 to 46 |
| CDH23-12R3a | TAGTAAAACTAGATAATTACACTACCGACTG | 32 | | | |
| CDH23-12F4 | ACACAGAGGTGCAGAGAGGTGACATAACTTCC | 32 | 6815 bp | 12F4/12R6 | 47 to 53 |
| CDH23-12R6 | TAGCACAGCCCATATAGTAACCACTGTTCAATAC | 34 | | | |
| CDH23-13F | CTTGGACACCCATGATGTCTTGGGGGGTGGGA | 32 | 12462 bp | 13F/13R | 53 to 68 |
| CDH23-13R | GTGACCCTCCTTACCTTGTCCTTAGATGCTTAACATT | 37 | | | |

TABLE 3
GJB2 primers

| | | | | | |
|---|---|---|---|---|---|
| primer name | primer sequence | bases | product size | primer pair | exons |

| | | | | | |
|---|---|---|---|---|---|
| GJB2-1F | AACCTTAGTCCTUGGCACATTGTTGAA | 27 | 6478 bp | 1F/1R2 | 1 to 2 |
| GJB2-1R2 | AACACCACATTGTCCATAGACTGATATG | 28 | | | |
| GJB2-1F2 | AGTCAATGCTAATAATGGTGGCAATCACG | 29 | 7156 bp | 1F2/1R2 | 2 |
| GJB2-1R2 | AACACCACATTGTCCATAGACTGATATG | 28 | | | |

TABLE 4
GJB6 primers

| | | | | | |
|---|---|---|---|---|---|
| primer name | primer sequence | bases | product size | primer pair | exons |

| | | | | | |
|---|---|---|---|---|---|
| GJB6-1F | TATGAGAAGGCTGGATCACCCAGAAAGACTG | 31 | 11,112 bp | 1F/1R | all 4 exons |
| GJB6-1R | TGAGGACATCATCCTAGTGTCGTACAAGTGG | 31 | | | |
| GJBB-2F-1 | TGTGTTCCTGGATTAATGCAAACAGC | 26 | 2361 bp | 2F-1/2R-2 | all 4 exons |
| GJB6-2R-2 | GGACATCATCCTAGTGTCGTACAAGT | 26 | | | |
| GJB6-2F-2 | AGCCAATCTGGTGTAATGGATCAGAC | 26 | 2383 bp | 2F-2/2R-1 | all 4 exons |
| GJB6-2R-1 | AGTGCTCTGTAGGCTGCTAAACTTAG | 26 | | | |

TABLE 5
KCNE1 primers

| | | | | | |
|---|---|---|---|---|---|
| primer name | primer sequence | bases | product size | primer pair | exons |

| | | | | | |
|---|---|---|---|---|---|
| KCNE-1F | GAAAGAGGCATGGAGAGTGAT | 21 | 1719 bp | 1F/1R1 | 1 to 2 |
| KCNE-1R1 | CTGAAGCTCACTGACGTCTGT | 20 | | | |
| KCNE-1F1 | CATGGATACCAAGAGACAACT | 21 | 1724 bp | 1F1/1R | |
| KCNE-1R | AGGATCACCTTCCTTGATTC | 20 | | | |
| KCNE-2F | TCCATTAAGGAAGGACCTTG | 20 | 437 bp | 2F/2R | 3 |
| KCNE-2R | TAAACATTCAGCGAATGCAG | 20 | | | |
| KCNE-3F1 | AACCAGTCTGACTAGTCTTGCATAAGCT | 28 | 4893 bp | 3F1/3R2 | 4 |
| KCNE-3R2 | GAGTCTGTTTATGCTTCTGTCAGGTGT | 28 | | | |

TABLE 6
KCNQ1 primers

| | | | | | |
|---|---|---|---|---|---|
| primer name | primer sequence | bases | product size | primer pair | exons |

| | | | | | |
|---|---|---|---|---|---|
| KNQ1-1F1 | GGTAAATGCACACTGGAACG | 20 | 1168 bp | 1F1/1R1 | 1 |
| KNQ1-1R1 | AGGATTCACACCTGGACTAC | 20 | | | |
| KNQ1-2F | ATCCACGTGGCAGCATGTGTTG | 22 | 564 bp | 2F/2R | 2 |
| KNQ1-2R | CTTTCAGACCACCAGCTCCAGGTT | 24 | | | |
| KNQ1-3F | ATGAGCTGAAGCTGCTCAGCCTTC | 24 | 2709 bp | 3F/3R | 3 to 6 |
| KNQ1-3R | TCCAAGCACAGGTTTGTGGACAG | 23 | | | |
| KNQ1-4F | GCTCTGTTCCTGGTGCTTTCGCCGAGT | 27 | 6183 bp | 4F/4R1 | 7 to 10 |
| KNO1-4R1 | GACAGGTCTGCCATCCAATCGTCAGGT | 27 | | | |
| KNQ1-5F1 | GACACTGAGGTGTCAGGCACTT | 22 | 532 bp | 5F1/5R1 | 11 |
| KNQ1-5R1 | AGGATCATGTTCCCAGGCTCA | 21 | | | |
| KNQ1-6F | TTGCTATGGCTGCCATGTGTCAGCAGCATAG | 31 | 9883 bp | 6F/6R | 12 to 15 |
| KNQ1-6R | TCTGCCACCCTCCACTCAGGACACAGCCAG | 30 | | | |
| KNQ1-7F | TTGCAGACATAGGGTGCACACGTGC | 25 | 1589 BP | 7F/7R | 16 |
| KNQ1-7R | AACAGGAGCGACGTCGCTAAGCTAG | 25 | | | |

TABLE 7
MYO7A primers

| | | | | | |
|---|---|---|---|---|---|
| primer name | primer sequence | bases | product size | primer pair | exons |

| | | | | | |
|---|---|---|---|---|---|
| MYO7A-1F | AGCACATCAGTGATTAAGTCAGG | 23 | 822 bp | 1F/1R | 1 |
| MYO7A-1R | GATTCGATGGACAACATGCTCCT | 23 | | | |
| MYO7A-2F | TTGGGAATCTCTGAATGACAGTG | 23 | 434 bp | 2F/2R | 2 |
| MYO7A-2R | GGTTTGGAAGCCTAGGCAGGAA | 22 | | | |
| MYO7A-3F | GAGAGGCCTTGGCTCTCTCTGA | 22 | 628 bp | 3F/3R | 3 |
| MYO7A-3R | TCTCTAACACCATGCAGAGTGG | 22 | | | |
| MYO7A-4F8 | CTGATGTCCAGATTCCTGCTAGT | 23 | 2863 bp | 4F8/4R8 | 4 |
| MYO7A-4R8 | ACCTCCAGCATTTATTCATGCCATG | 25 | | | |
| MYO7A-5F | AGAAGGAAATCTAGGCTTAGAGACTCCACCTCCC | 34 | 7707 bp | 5F/5R | 5 to 14 |
| MYO7A-5R | GCATATGATTCCACTTATATGAGGTACCTAGAAT | 34 | | | |
| MYO7A-6F | TGGATGTGGTGGAACTAGGTGG | 22 | 488 bp | 6F/6R | 15 |
| MYO7A-6R | AACCGATCCCTGACCGGTTCTG | 22 | | | |
| MYO7A-7F1a | AGAGGTGGTAACTTTGGAAGTCCTGG | 26 | 7573 bp | 7F1a/7R1a | 16 to 21 |
| MYO7A-7R1a | GGTATGTGCACTCCTCAGAGCAGGCATA | 28 | | | |
| MYO7A-7F1d | TGGTCAGATGGATAGATGGCATCACCTC | 28 | 4102 bp | 7F1d/7R1a2 | 16 to 18 |
| MYO7A-7R1a2 | ATCACATCTTGCTGATGAGGAAATGCAGG | 29 | | | |
| MYO7A-7F1e | TCACAGTCTGGTGGCATAGTACCTAAATTG | 30 | 4128 bp | 7F1e/7R1a1 | 16 to 18 |
| MYO7A-7R1a1 | CTCCCAGGTTGTAGATGATCTCAAACAC | 28 | | | |
| MYO7A-7F21a | TGCAGCTCCTGATCTAGGAT | 20 | 591 bp | 7F21a/7R21a | 21 |
| MYO7A-7R21a | AGAGCAGGCATAACTGCAG | 21 | | | |
| MYO7A-7F21b | ATTAGAGATCTCAGACAGGGTG | 22 | 898 bp | 7F21b/7R21b | 21 |
| MYO7A-7R21b | AACTGGGCATGACTTTGATAGG | 22 | | | |
| MYO7A-7F2a | ACCTCAGTCACTCTTGGGAATCTCTG | 26 | 3361 bp | 7F2a/7R2a | 22 to 26 |
| MYO7A-7R2a | TAGAAGTGTATTCCCTCTCAGCTGTG | 26 | | | |
| MYQ7A-8F | TGCAGGGTATCGAGGAGGTGGC | 22 | 620 bp | 8F/8R | 27 |
| MYO7A-8R | TGCAATATCTCCAAGGGATGCC | 22 | | | |
| MYO7A-9F1 | GGCCCCTTAAGTATTCACACATTACAGAAATA | 32 | 11,772 bp | 9F1/9R3 | 28 to 35 |
| MYO7A-9R3 | GTTGAAACTTGATCTCCCAGTGTTGGCAGTGG | 32 | | | |
| MYO7A-10F | CGAGGTGGAAGGAGTCTGGGAGGCCCGCTCACAA | 34 | 8018 bp | 10F/10R | 36 to 44 |
| MYO7A-10R | AGACACATAATAGAGGCTCAACATGCAAGCTTCC | 34 | | | |
| MYO7A-11F | GGCCATGCACTCCAACTGCCAACTGCTGAGTCT | 33 | 4555 bp | 11F/11R | 45 to 49 |
| MYO7A-11R | TCACCTCCCAGCCTGATGTCCAGCACTTCCTCC | 33 | | | |

TABLE 8
OTOF primers

| | | | | | |
|---|---|---|---|---|---|
| primer name | primer sequence | bases | product size | primer pair | exons |

| | | | | | |
|---|---|---|---|---|---|
| OTOF-1F | TGGTAGCACATAAGCCTCTG | 20 | 1001 | 1F/1R | 1 |
| OTOF-1R | ATCACAATGGCCAGTCAGTC | 20 | | | |
| OTQF-2F | TCCTAACATGGAACTCATGG | 20 | 451 | 2F/2R | 2 |
| OTQF-2R | TTACCACCTCCTTCAGGAAG | 20 | | | |
| OTQF-3F | CCAACATCTCTGAGCACCAT | 20 | 786 | 3F/3R | 3 |
| OTOF-3R | TGAGTGTCTGAGATCAGGC | 19 | | | |
| OTOF-4F | ACAAACAACCATCCACAGTGGG | 22 | 3197 | 4F/4R | 4 to 5 |
| OTQF-4R | TCTGAGAAAGGCAGGAGATCTAG | 23 | | | |
| OTOF-5F | AAAGACAAGTCAGGCTTTGAGCAC | 24 | 2937 | 5F/5R | 6 to 8 |
| OTOF-5R | TATGAAGTCCAATACTGAACATG | 23 | | | |
| OTOF-6F | TGTGGTAGTGCATGCCTGTAATCC | 24 | 6513 | 6F/6R | 9 to 11 |
| OTOF-6R | ATGGCTGTGTGTACTAACAGTCGC | 24 | | | |
| OTOF-7F1a | AGCTCCAGAGGACCTCAGACTCTATC | 26 | 4152 | 7F1a/7R1a | 12 to 25 |
| OTOF-7R1a | TGAGGTATGACTCCTCAGGTAGACAG | 26 | | | |
| OTOF-7F2a | CCTGCTTCCATGGATATCCAGGCT | 24 | 5373 | 7F2a/7R2a | 16 to 25 |
| OTOF-7R2a | CTCAGTCTGTAGGAGACAGGAGGTGA | 26 | | | |
| QTOF-7F2e | CTGTGGAGATCGTAGACACCTCCAA | 25 | 1791 | 7F2e/7R2e | 16 to 18 |
| OTOF-7R2e | ACTAGAGGTGGCTCCTGTCCTTGTC | 25 | | | |
| OTOF-7F2f | TAACTACACGCTGCTGGATGAGCATC | 26 | 1784 | 7F2f/7R2d | 16 to 18 |
| OTOF-7R2d | AGACCAGCTTTGTGTGTTCCAGGGAAG | 27 | | | |
| OTOF-7F2e | CTGTGGAGATCGTAGACACCTCCAA | 25 | 3315 | 7F2e/7R2i | 16 to 20 |
| OTOF-7R2i | CTCTGTAGATTCTTCCTCATCTGCCC | 26 | | | |
| OTOF-7F2f | TAACTACACGCTGCTGGATGAGCATC | 26 | 3404 | 7F2f/7R2i | 16 to 20 |
| OTOF-7F2m | TGATCAACAGGGAGGAGGCATTT | 23 | 955 | 7F2m/7R2m | 19 to 20 |
| OTOF-7R2m | CTGCCCCCTCCAGCACCTTA | 20 | | | |
| QTOF-7F2n | CCTAGCGAGAGCTCCCAG | 18 | 542 | 7F2n/7R2n | 19 to 20 |
| OTOF-7R2n | GACAGCTCGGGCCATGAC | 18 | | | |
| OTOF-7F3f1 | TGGGCAGATGAGGAAGAATCTACAGAGC | 28 | 2838 | 7F3f1/7R3a1 | 21 to 25 |
| OTOF-7R3a1 | flACCACAGCGCCATGAGTTGTTGTAAG | 28 | | | |
| OTOF-7R3b1 | ACATGAGGTCCTCCTACCTCTAGTCCAG | 28 | 2697 | 7F3f1/7R3b1 | 21 to 25 |
| OTOF-7F-A | CTGTGGAGATCGTAGACACCTCCAACCCTGAGCT | 34 | 16,256 | 7F-A/7R-A | 16 to 39 |
| OTOF-7R-A | CAGATAGCCTCTCTACCTCACTGGGA1TTGGACA | 34 | | | |
| OTOF-8F5 | TAAGGACCAAACGAGATCACAGGTGTGGA | 29 | 10127 | 8F5/8R6 | 26 to 39 |
| OTOF-8R6 | AGCCTCTCTACCTCACTGGGATTTGGACA | 29 | | | |
| OTOF-8R7 | CGAGTCACTAGAAGTAGGATCTTGGTTTGT | 30 | 10181 | 8F5/8R7 | 26 to 39 |
| OTOF-8R4 | GGTTTGTTCTACCTCACTGGGATTTGGACA | 30 | 10128 | 8F5/8R4 | 26 to 39 |
| OTOF-9F1 | GTAGACAGGTGATGGCATAGAGGCTTCT | 28 | 7106 | 9F1/9R1 | 40 to 47 |
| OTOF-9R1 | TGGTACTGAATCTGCCAGCCTAGAGAAC | 28 | | | |
| OTOF-9F2 | AGGCACTTCCCAGAGAAGCAGAGAATTG | 28 | 7759 | 9F2/9R9 | 40 to 47 |
| OTOF-9R9 | TGTGGCTGAATCTCTTTAAAGAGGTCAGG | 29 | | | |

TABLE 9
SLC26A4 sequences

| | | | | | |
|---|---|---|---|---|---|
| primer name | primer sequence | bases | product size | primer pair | exons |

| | | | | | |
|---|---|---|---|---|---|
| SLC-1F | TCAGAGAATTTGCATCAGGGTTCTC | 25 | 3665 | 1F/1R | 1 to 3 |
| SLC-1R | TAAGCAACCATCTGTCACAGACC | 23 | | | |
| SLC-2F2 | TGGAACCATTGTAAGTTGAGGACTT | 25 | 3225 | 2F2/2R4 | 4 to 6 |
| SLC-2R4 | GAGATGAGGTCTCACGTCTCAAACT | 25 | | | |
| SLC-3F | ATCAACTGGGAGTTTCAGGTTTATCAGCC | 29 | 7618 | 3F/3R | 7 to 10 |
| SLC-3R | AAGGCAAATTGTCCTGCTAAGCTCGGTG | 28 | | | |
| SLC-4F | AATGAGACCATGTGCTACAAGTACGAAGTG | 30 | 11306 | 4F/4R | 11 to 18 |
| SLC-4R | TTTGTTCACTCTTACCTAGGTGAGAGCCTG | 30 | | | |
| SLC-5F4 | GATCGTCCACAAGGTTGACTACGACCAGT | 28 | 9069 | 5F4/5R6 | 19 to 21 |
| SLC-5R6 | TCATTGATTCTCACCTCACAGATCTAAGC | 29 | | | |

TABLE 10
USH2A sequences

| | | | | | |
|---|---|---|---|---|---|
| primer name | primer sequence | bases | product size | primer pair | exons |

| | | | | | |
|---|---|---|---|---|---|
| USH2A-1F | TAGGATAAGGTGTACTGCTACTT | 23 | 5085 | 1F/1R | 1 to 3 |
| USH2A-1R | GAAGACAAATCCTTGTGTTTAACCA | 25 | | | |
| USH2A-2F | AACACATGGAGATATCACTGAGC | 23 | 699 | 2F/2R | 4 |
| USH2A-2R | CCTAAATCCAATGACAAGTGTCCTT | 25 | | | |
| USH2A-3F1 | CTTAAGTCCTACAGTGTCCATGGAGATA | 28 | 7298 | 3F1/3R1 | 5 to 9 |
| USH2A-3R1 | CATCAGTGATGTGTTAAAGGTTATATTC | 28 | | | |
| USH2A-4F | TCACTGATATGTGCTTTACTTCTGG | 25 | 3302 | 4F/4R | 10 to 11 |
| USH2A-4R | AGGATTTCCTGGCAAATGCAGTCTTC | 26 | | | |
| USH2A-5F | GTCTTGTACCTAATGAGCAAATTATCT | 27 | 4954 | 5F/5R | 12 to 13 |
| USH2A-5R | GCATTGTATGGATATTCAACTCAAATT | 27 | | | |
| USH2A-6F1 | GAATTAGTGCCTTGGTAGA | 19 | 378 | 6F1/6R | 14 |
| USH2A-6F2 | GTATTGGGAATTAGTGCCTT | 20 | 386 | 6F2/6R | 14 |
| USH2A-6R | CAGAAGTTATTGCTTTGCAACT | 22 | | | |
| USH2A-7F | CTCTACAATGCTATTGGTAGGTGTAACTTA | 30 | 10458 | 7F/7R | 15 to 16 |
| USH2A-7R | CACAACAGCATTTATCCTCAATGTCAAAGA | 30 | | | |
| USH2A-8F | AGCAGTTAGCAATGATTCTTCACCAACTTGTG | 32 | 10312 | 8F/8R | 17 to 20 |
| USH2A-8R | CCTGGAGTCACGCTACAACTAATTACATTTCT | 32 | | | |
| USH2A-9F | TTCCTAGAGCCATACAGATACTTG | 24 | 1826 | 9F/9R | 21 |
| USH2A-9R | GCTGAATGGAAACGGATGCTATT | 23 | | | |

The following examples disclose various applications of the present invention and are not intended to be limiting. These examples can be used in conjunction with conventional pediatric screening methods or as a primary screening tool.

### Example 3

### “Resequencing” Array

Prior to implementation of the array in the screening of pediatric patients, a “resequencing” microarray is produced for mutational analysis and to perform initial characterization of the array's abilities to detect and perform sequence analysis of the labeled PCR products. One such “resequencing” microarray is prepared as follows:

An array is constructed such that each of a possible 60,000 positions to be sequenced are represented by 8 different oligonucleotides; 4 for each possible base on both upper and lower strand. Configured in this way, the reliability of the sequence read is extremely high (>99.9999%). High density VDAs are fabricated using standard photolithographic and solid phase DNA synthesis. Each of the 300,000 features are 24×20 μm in size. A feature consists of ˜10⁶ copies of an approximate 25-bp long oligonucleotide probe of a defined sequence. To utilize the array, the PCR products are hydrolyzed to an average size of about 75 to about 250 bp, subjected to biotinylation, and hybridized to the chip using the standard antibody detection method for the detection of hybridization intensity analysis.

### Example 4

### Validation Study

After informed consent is obtained, GJB2 mutant DNA is compared between analysis performed by microarray and sequencing in 10 subjects (˜6×10⁵ bp). The microarray results are compared for heterozygous and homozygous call accuracy compared to sequencing. This study provides data to ensure that the microarray tool has been constructed according to the desired specifications. In addition, a large-scale validation study is performed that includes the sequencing of the PCR products from a cohort of hearing loss subjects on both a conventional sequencer and the fabricated array. In preferred embodiments, about 100 subjects, or more, are sampled for such validations studies.

### Example 5

### First Generation Variation Detection Arrays (VDA)

A VDA is constructed containing capture nucleotide sequences representing the following candidate genes. The capture nucleotide sequences on the array include the mutants for the specific gene(s) to be screened for.

| Genes | Phenotype(s) | No. of mutants |
|---|---|---|
| GJB2 | DFNB1 | >50 |
| | | (autosomal recessive) |
| | DFNA3 | 6 |
| | | (autosomal dominant) |
| | PPK | |
| GJB6 | DFNB1 | |
| | DFNA3 | |
| | Clouston's | |
| SLC26A4 | DFNB4/EVA/PDS | >50 |
| OTOF | DFNB9 | 3 |
| | AN | 2 |
| MYO7A | DFNB2 | 4 |
| | DFNA11 | 1 |
| | USH18 | |
| CDH23 | DFNB12 | 7 |
| | USH1D | 7 |
| USH2A | USH2A | >10 |
| KCNQ1 | JLN | >30 |
| KCNE1 | JLN | |
| PAX3 | WS1 | many (indefinite) |

A blood sample is collected from a pediatric patient and DNA is isolated from the blood sample using a commercially avaiable kit for that purpose (Qiagen, Inc.). Briefly, following the commercial protocol, a 200 ì L sample of whole blood drawn from a patient is placed in a microcentrifuge tube with 20 ì L of Qiagen protease, 200 ì L of “Buffer AL”, a detergent solution, and 4 ì L of a Qiagen RNase stock solution, to lyse the cells and solubilize the cellular debris released during cell lysis. After heating the tube at 56° C. for 10 minutes, the tube is briefly spun in a microcentrifuge, 200 ì L of 100% ethanol is added to the tube, the contents are mixed with brief vortexing and briefly spun in a microcentrifuge in order to collect all of the tube contents at the bottome of the tube. The contents of the tube are then placed in a QIAamp spin column. These columns contain a resin that binds nucleic acids under mildly acidic pH conditions. By spinning the column in a microcentrifuge for one minute at 8000 RPM, the solution is pulled through the resin and the chromosomal DNA from the blood sample is bound to the resin. The filtrate is discarded and the resin with the attached DNA is then washed by applying 500 ì L of wash buffer AW1 and spinning the column for 1 minutes at 8000 RPM. The wash filtrate is discarded and 500 ì L of wash buffer AW2 is added to the column. The column is spun for 3 minutes at 14,000 RPM and the filtrate discarded. An additional spin cycle for 1 minute at 14,000 RPM is performed to ensure full removal of the wash buffer from the column. To elute the sample, 200 ì L of Buffer AE, which has a mildly basic pH, is added to the resin and allowed to incubate for 1 minute at room temperture. The incubation is followed by a short spin in the microcentrifuge, producing a highly purified DNA sample with a typical yield of 6 ì g of DNA in about 200 ì L of buffer.

Certain portions of the genomic DNA sample are amplified with long PCR to amplify those regions of unique, non-repetitive sequence that contain the genetic loci of interest and create a sufficient amount of DNA for use in the microarray screening protocol. Following a protocol as described in Cutler, et al (ibid), long PCR primers are designed using published human genomic sequence and the Amplify 1.2 primer designing software program. The primers are 30 to 32 bases in length, to ensure that they bind uniquely to those blocks of genomic sequence that are to be amplified, have a GC content of between 45% and 60% and end with a pyrimidine nucleotide. PCR amplification reactions are carried out with TaKaRa LA Taq enzyme (TaKaRa Biomedicals, Inc.) with the addition of DMSO to the manufacturer's standard PCR mixture to assist in the amplification of GC-rich genomic sequence. An annealing temperture of 68° C. is used to reduce mispriming and ensure high fidelity of the PCR. The reactions contain 100 ng of genomic DNA as a template and generate fragments of amplified genomic sequence of about 6 to 7 kilobases in length. Successful amplification of genomic sequences is verified by analyzing some of the product from each reaction on a 1% agarose gel. The bands of amplified DNA are compared to a large molecular weight DNA ladder standard to verify size and estimate the yield of the PCR reactions.

This DNA sample is analyzed using the array of the invention and standard array analysis protocols. For an example of the use of microarrays in the detection to mutations within genomic DNA samples from humans, see Cutler et al (ibid), as well as Hacia, J et al (1998) Genome Res 8:1245-1258, which is hereby incorporated by reference in its entirety. Briefly, before application of the DNA to the microarray of the invention, the amplified genomic DNA is subjected to brief digestion with DNAse I, in order to create fragments of genomic DNA that are a more suitable size for use with a microarray of the invention. Genomic DNA, DNAseI and acetylated bovine serum albumin (BSA) (both products obtained from Pharmacia Biotech, Inc.) are place in snap-top tubes and incubated in a 37° C. water bath for 15 minutes, followed by an incubation at 99° C. for 15 minutes to inactivate the enzyme. The fragments undergo labeling with biotin using 1 mM Biotin-N-6-ddATP (NEN Life Sciences) and 15 U/ì L rTdT enzyme (Gibco BRL). Labeling takes place during a 37° C. incubation for 90 minutes, which is followed by a 99° C. incubation for 15 minutes to inactivate the enzyme. Analysis of the fragmented and labeled DNA with the microarray of the invention takes place in four steps: pre-hybridization, hybridization, washing and scanning. The pre-hybridization involves incubating the array of the invention with a 10 mM Tris solution (pH 7.8) containing 3M TMACL (tetramethyl ammonium chloride) and 1% Triton X-100 detergent for 5 minutes. Hybridization of the labeled DNA takes place using a 10 mM Tris solution (pH 7.8) containing the DNA sample (100 ì g/ml), 3M TMACL, 500 ì g/ml BSA, 0.01% Tween 20 detergent; the array of the invention is incubated with this solution for 16h at 44° C. under rotation at 60 rpm. After the hybridization period, the sample solution is removed from the array and the array is washed twice for 10 minutes at a time at 25° C. in a standard wash buffer of 6×SSPE and 0.01% Tween 20. The array is then stained with a solution of 5 ì g/mL SAPE, 6×SSPE, 0.01% Tween 20 and 2 mg/ml BSA for 15 minutes. An additional wash cycle is followed by staining with phycoerythrin-strepavidin conjugate (Molecular Probes, Inc.) for 5 minutes at room temperture. After a wash cycle, data is obtained from the array with a scanning confocal microscope equipped with a 488-nm argon laser (Gene Chip Scanner [Affymetrix, Inc.]). The data is visualized and analyzed using software from Affymetrix (GeneChip Software).

### Example 6

### Second Generation VDA

A VDA is constructed containing capture nucleotide sequences representing the following candidate genes. The capture nucleotide sequences on the array include the mutants for the specific gene(s) to be screened for.

| Genes | Phenotype(s) | No. of mutants |
|---|---|---|
| PCDH15 | USH1F | Uncommon |
| GJA1 | | ″ |
| TECTA | DFNB21 | ″ |
| | DFNA8/12 | |
| TMIE | DFNB6 | ″ |
| | ?USH2B? | ″ |
| Harmonin | USH1C | ″ |
| | ?DFNB18 | |

Samples are collected from pediatric patients and screened using the Second Generation VDA.

### Example 7

### Third Generation VDA

A VDA is constructed containing capture nucleotide sequences representing the following candidate genes. The capture nucleotide sequences on the array include the mutants for the specific gene(s) to be screened for.

| Genes | Phenotype(s) | No. of mutants |
|---|---|---|
| Prestin | DFNB | Rare |
| TMPRSS3 | DFNB8/10 | ″ |
| OTOA | DFNB22 | ″ |
| STRC | DFNB16 | ″ |
| MITF | WS2 | ″ |
| MYO15 | DFNB3 | ″ |
| TMC1 | DFNB7/11 | ″ |
| CLD14 | DFNB29 | ″ |
| USH3 | USH3 | ″ |
| COL4A5 | Alport | ″ |
| COL4A3 | Alport | ″ |
| COL4A4 | Alport | ″ |

Samples are collected from pediatric patients and screened using the Third Generation VDA.

### Example 8

### Polymorphisms of DFNB 1 VDA

An array is constructed containing capture nucleotide sequences containing primers directed towards mutant sequences that cause DFNB1 and their normal counterparts. Samples within a target population and/or target populations are collected from pediatric patients and screened using this array. The prevalence of particular genetic mutations that cause DFB 1 in the target population is revealed in the microarray data.

Target populations can include screening various Caucasian populations to identify which mutants of DFNB1 are associated with the Caucasian population. The same screening can be applied to any population group in order to ascertain which mutations can be representative of certain target populations.

### Example 9

### Screening of Newborns for Genetic Mutations Associated with Hearing Loss

Gene chip microarrays are constructed according to the methods outlined above. Normal and mutant genetic sequences to be screened include the genes listed above in examples 4 through 6. Normal sequences throughout the genes being surveyed are sampled among the capture probe sequences in order to screen for possible novel missense, nonsense and deletion mutations in genes associated with hearing loss.

DNA samples are collected from infants and amplified, labeled DNA samples are prepared using readily available commercial kits for those purposes. The DNA samples are applied to the microarray chips, the DNA is interrogated and the data processed, according to Affymetrix protocols.

Infants who are identified as carrying alleles associated with hearing loss are tested with physiological methods to confirm the hearing impairment. Using information gained from the microarray DNA analysis, a habilitation program is created, tailored to the individual hearing needs of the infant according to his/her specific impairment.

Exemplary Applications

The diagnostic array of the present invention for determining the etiology of genetic hearing loss in infants can be used in conjunction with conventional newborn hearing screening methods or can be used as a replacement of some aspects of conventional newborn hearing screening methods.

The diagnostic array of the present invention can be used to compare polymorphisms within the candidate genes, accounting for the known mutations and attempting to discover new mutations of the candidate genes as exemplified in Example 8. Target populations can be screened and comparisons within the populations and to other target populations can be determined in order to better identify which types of mutations arise in certain target populations for certain target genes.

Arrays containing capture nucleotide sequences can be directed toward specific ethnicities, specific populations and the like. This enables “designer” arrays to be designed in order to fit the needs of newborn hearing screening methods in the United States, in Europe, in Asia, in Southeast Asia, in regions of the Middle East, etc., to account for the genetic variability of these genes associated with pediatric hearing loss within these populations.

In the future, arrays contemplated by the present invention can be used to detect early on disorders relating to hearing loss and/or disorders that include hearing loss as a symptom of the disorder. This information can be used to develop recombinant genes that can be applied to genetic therapy of the diagnosed disorder.

### Conclusion

The Examples described above are set forth solely to assist in the understanding of the invention. Thus, those skilled in the art will appreciate that the present invention can provide for a microarray and diagnostic method for identifying genes associated with pediatric hearing loss. The candidate genes, capture nucleotides sequences and arrays described herein are presently representative of certain embodiments and are exemplary and are not intended as limitations on the scope of the invention. Changes therein and other uses will occur to those skilled in the art which are encompassed within the spirit of the invention. It will be readily apparent to one skilled in the art that varying substitutions and modifications can be made to the invention disclosed herein without departing from the scope and spirit of the invention. Thus, it should be understood that although the present invention has been specifically disclosed by preferred embodiments and optional features, modification and variation of the concepts herein disclosed can be resorted to by those skilled in the art, and that such modifications and variations are considered to be falling within the scope of the invention, which is limited only by the following claims.

1. A method for diagnosing a cause or risk factor for hearing loss, comprising:
obtaining a sample from a patient;
amplifying genetic sequences found in said sample;
screening the amplified genetic sequences for the presence or absence of alleles of at least 5 loci associated with a risk for hearing loss to obtain a result of the screening; and
making a diagnosis based upon the result.

2. The method of claim 1, wherein said amplifying is performed by polymerase chain reaction.

3. The method of claim 1, wherein said amplifying involves the use of a primer sequence found in Tables 2-10.

4. The method of claim 1, wherein said genetic sequences comprise sequences found in genes selected from the group consisting of CDH23, MYO7A, OTOF, SLC26A4, USH2A, KCNQ1, KCNE1, GJB2 and GJB6.

5. The method of claim 4, wherein said genetic sequences comprise sequences of at least two adjacent exons.

6. The method of claim 5, wherein said multiple adjacent exons are selected from the group consisting of CDH23 exons 2-3, CDH23 exons 4-6, CDH exons 7-9, CDH23 exons 10-11, CDH23 exons 12-13, CDH23 exons 14-16, CDH23 exons 17-21, CDH23 exons 22-27, CDH23 exons 28-31, CDH23 exons 32-36, CDH23 exons 37-43, CDH23 exons 44-46, CDH23 exons 47-53, CDH23 exons 53-68, GJB2 exons 1-2, GJB6 exons 1-4, KCNE1 exons 1-2, KCNQ1 exons 3-6, KCNQ1 exons 7-10, KCNQ1 exons 12-15, MYO7A exons 5-14, MYO7A exons 16-21, MYO7A exons 16-18, MYO7A exons 22-26, MYO7A exons 28-35, MYO7A exons 36-44, MYO7A exons 45-49, OTOF exons 4-5, OTOF exons 6-8, OTOF exons 9-11, OTOF exons 12-25, OTOF exons 16-25, OTOF exons 16-18, OTOF exons 16-20, OTOF exons 19-20, OTOF exons 21-25, OTOF exons 16-39, OTOF exons 26-39, OTOF exons 40-47, SLC26A4 exons 1-3, SLC26A4 exons 4-6, SLC26A4 exons 11-18, SLC26A4 exons 19-21, USH2A exons 1-3, USH2A exons 5-9, USH2A exons 10-11, USH2A exons 12-13, USH2A exons 15-16 and USH2A exons 17-20.

7. The method of claim 4, wherein said genetic sequences comprise a single exon.

8. The method of claim 7, wherein said single exon is selected from the group consisting of GJB2 exon 2, KCNE1 exon 3, KCNE1 exon 4, KCNQ1 exon 1, KCNQ1 exon 2, KCNQ1 exon 11, KCNQ1 exon 16, MYO7A exon 1, MYO7A exon 2, MYO7A exon 3, MYO7A exon 4, MYO7A exon 15, MYO7A exon 21, MYO7A exon 27, OTOF exon 1, OTOF exon 2, OTOF exon 3, USH2A exon 4, USH2A exon 14 and USH2A exon 21.

9. A method for diagnosing a cause or risk factor for hearing loss, comprising:
obtaining a sample from a patient;
screening said sample for the presence or absence of alleles of at least 5 loci associated with a risk for hearing loss to obtain a result of the screening, wherein said loci comprise sequences found in genes selected from the group consisting of CDH23, MYO7A, OTOF, SLC26A4, USH2A, KCNQ1, KCNE1, GJB2 and GJB6; and
making a diagnosis based upon the result.

10. The method of claim 9, wherein the amount of genetic material in said sample is augmented before or during said screening.

11. The method of claim 10, wherein said augmentation is amplification performed by polymerase chain reaction.

12. The method of claim 10, wherein said augmentation involves the use of a primer sequence found in Tables 2-10.

13. The method of claim 9, wherein said sequences found in genes selected from the group consisting of CDH23, MYO7A, OTOF, SLC26A4, USH2A, KCNQ1, KCNE1, GJB2 and GJB6 comprise sequences of at least two adjacent exons.

14. The method of claim 13, wherein said multiple adjacent exons are selected from the group consisting of CDH23 exons 2-3, CDH23 exons 4-6, CDH exons 7-9, CDH23 exons 10-11, CDH23 exons 12-13, CDH23 exons 14-16, CDH23 exons 17-21, CDH23 exons 22-27, CDH23 exons 28-31, CDH23 exons 32-36, CDH23 exons 37-43, CDH23 exons 44-46, CDH23 exons 47-53, CDH23 exons 53-68, GJB2 exons 1-2, GJB6 exons 1-4, KCNE1 exons 1-2, KCNQ1 exons 3-6, KCNQ1 exons 7-10, KCNQ1 exons 12-15, MYO7A exons 5-14, MYO7A exons 16-21, MYO7A exons 16-18, MYO7A exons 22-26, MYO7A exons 28-35, MYO7A exons 36-44, MYO7A exons 45-49, OTOF exons 4-5, OTOF exons 6-8, OTOF exons 9-11, OTOF exons 12-25, OTOF exons 16-25, OTOF exons 16-18, OTOF exons 16-20, OTOF exons 19-20, OTOF exons 21-25, OTOF exons 16-39, OTOF exons 26-39, OTOF exons 40-47, SLC26A4 exons 1-3, SLC26A4 exons 4-6, SLC26A4 exons 11-18, SLC26A4 exons 19-21, USH2A exons 1-3, USH2A exons 5-9, USH2A exons 10-11, USH2A exons 12-13, USH2A exons 15-16 and USH2A exons 17-20.

15. The method of claim 9, wherein said sequences found in genes selected from the group consisting of CDH23, MYO7A, OTOF, SLC26A4, USH2A, KCNQ1, KCNE1, GJB2 and GJB6 comprise a single exon.

16. The method of claim 15, wherein said single exon is selected from the group consisting of GJB2 exon 2, KCNE1 exon 3, KCNE1 exon 4, KCNQ1 exon 1, KCNQ1 exon 2, KCNQ1 exon 11, KCNQ1 exon 16, MYO7A exon 1, MYO7A exon 2, MYO7A exon 3, MYO7A exon 4, MYO7A exon 15, MYO7A exon 21, MYO7A exon 27, OTOF exon 1, OTOF exon 2, OTOF exon 3, USH2A exon 4, USH2A exon 14 and USH2A exon 21.

17. A diagnostic hearing loss microarray comprising at least 5 sequences that are indicative of presence or absence of an allele associated with a risk for hearing loss, wherein said sequences are selected from the group consisting of genetic sequences from CDH23, MYO7A, OTOF, SLC26A4, USH2A, KCNQ1, KCNE1, GJB2 and GJB6.

18. The microarray of claim 17, wherein said sequences comprise multiple adjacent exons.

19. The microarray of claim 18, wherein said multiple adjacent exons are selected from the group comprising CDH23 exons 2-3, CDH23 exons 4-6, CDH exons 7-9, CDH23 exons 10-11, CDH23 exons 12-13, CDH23 exons 14-16, CDH23 exons 17-21, CDH23 exons 22-27, CDH23 exons 28-31, CDH23 exons 32-36, CDH23 exons 37-43, CDH23 exons 44-46, CDH23 exons 47-53, CDH23 exons 53-68, GJB2 exons 1-2, GJB6 exons 1-4, KCNE1 exons 1-2, KCNQ1 exons 3-6, KCNQ1 exons 7-10, KCNQ1 exons 12-15, MYO7A exons 5-14, MYO7A exons 16-21, MYO7A exons 16-18, MYO7A exons 22-26, MYO7A exons 28-35, MYO7A exons 36-44, MYO7A exons 45-49, OTOF exons 4-5, OTOF exons 6-8, OTOF exons 9-11, OTOF exons 12-25, OTOF exons 16-25, OTOF exons 16-18, OTOF exons 16-20, OTOF exons 19-20, OTOF exons 21-25, OTOF exons 16-39, OTOF exons 26-39, OTOF exons 40-47, SLC26A4 exons 1-3, SLC26A4 exons 4-6, SLC26A4 exons 11-18, SLC26A4 exons 19-21, USH2A exons 1-3, USH2A exons 5-9, USH2A exons 10-11, USH2A exons 12-13, USH2A exons 15-16 and USH2A exons 17-20.

20. The microarray of claim 17, wherein said sequences comprise a single exon.

21. The microarray of claim 20, wherein said single exon is selected from the group consisting of GJB2 exon 2, KCNE1 exon 3, KCNE1 exon 4, KCNQ1 exon 1, KCNQ1 exon 2, KCNQ1 exon 11, KCNQ1 exon 16, MYO7A exon 1, MYO7A exon 2, MYO7A exon 3, MYO7A exon 4, MYO7A exon 15, MYO7A exon 21, MYO7A exon 27, OTOF exon 1, OTOF exon 2, OTOF exon 3, USH2A exon 4, USH2A exon 14 and USH2A exon 21.

22. A kit for detecting a candidate gene responsible for hearing loss comprising:
a microarray of claim 17; and
buffers and components to be used with said microarray.

23. The kit of claim 22, wherein the microarray comprises a solid support comprising a plurality of capture nucleotide sequences bound to the solid support, wherein said capture nucleotide sequences are representative of regions of candidate genes for hearing loss, and wherein the support of the kit is adapted to be contacted with a sample from a patient comprising target nucleic acid sequences, and wherein the contacting permits hybridization under stringent conditions of a target nucleic acid sequence and a capture nucleotide sequence representative of regions of candidate genes for hearing loss.


SEQUENCE LISTING (163 sequences)

SEQ ID NO: 1 - DNA, 32 bp, Artificial Sequence
  Synthetic oligonucleotide primer sequence
cactgtgctatacccaggataggacaatgtta

SEQ ID NO: 2 - DNA, 32 bp, Artificial Sequence
  Synthetic oligonucleotide primer sequence
tcaggtggaagatgacctcaacctgtaagatc

SEQ ID NO: 3 - DNA, 28 bp, Artificial Sequence
  Synthetic oligonucleotide primer sequence
gataccatcatgacacactgtgacaagt

SEQ ID NO: 4 - DNA, 28 bp, Artificial Sequence
  Synthetic oligonucleotide primer sequence
gactcttcacctacaccatggtggtctg

SEQ ID NO: 5 - DNA, 33 bp, Artificial Sequence
  Synthetic oligonucleotide primer sequence
tatgtattcttcacactaaccctgtgagatatg

SEQ ID NO: 6 - DNA, 32 bp, Artificial Sequence
  Synthetic oligonucleotide primer sequence
tagccctcagagcctgagatgcctactggctc

SEQ ID NO: 7 - DNA, 25 bp, Artificial Sequence
  Synthetic oligonucleotide primer sequence
tgagtctttaatgcccagagaggag

SEQ ID NO: 8 - DNA, 25 bp, Artificial Sequence
  Synthetic oligonucleotide primer sequence
tgagatggagtcttactcttgttgc

SEQ ID NO: 9 - DNA, 24 bp, Artificial Sequence
  Synthetic oligonucleotide primer sequence
ccagaagctatggcccatcagagg

SEQ ID NO: 10 - DNA, 25 bp, Artificial Sequence
  Synthetic oligonucleotide primer sequence
gcaaccaagagtactgacagataca

SEQ ID NO: 11 - DNA, 34 bp, Artificial Sequence
  Synthetic oligonucleotide primer sequence
tgtaggtagaaggcgtgcaggagccagcagtcgc

SEQ ID NO: 12 - DNA, 34 bp, Artificial Sequence
  Synthetic oligonucleotide primer sequence
ggttcgagtgtttgctgctcagccttccgagtat

SEQ ID NO: 13 - DNA, 26 bp, Artificial Sequence
  Synthetic oligonucleotide primer sequence
ccaaaggagacgtgcgagaggaacat

SEQ ID NO: 14 - DNA, 25 bp, Artificial Sequence
  Synthetic oligonucleotide primer sequence
ttcctgagtagcccagagtgtcagg

SEQ ID NO: 15 - DNA, 33 bp, Artificial Sequence
  Synthetic oligonucleotide primer sequence
acctcagtcgagatgttgaggctccaggtgttc

SEQ ID NO: 16 - DNA, 32 bp, Artificial Sequence
  Synthetic oligonucleotide primer sequence
ctattgcaagagccagctcagagggacacaga

SEQ ID NO: 17 - DNA, 32 bp, Artificial Sequence
  Synthetic oligonucleotide primer sequence
gagggtttgatgaggaggaacccagtctccaa

SEQ ID NO: 18 - DNA, 33 bp, Artificial Sequence
  Synthetic oligonucleotide primer sequence
attaactcgctggctctaggatttcagtaagag

SEQ ID NO: 19 - DNA, 30 bp, Artificial Sequence
  Synthetic oligonucleotide primer sequence
gtaggatgcgtgaagggaaggaaaggaact

SEQ ID NO: 20 - DNA, 31 bp, Artificial Sequence
  Synthetic oligonucleotide primer sequence
gtgcacacagaaggagctcaaccaatgttgg

SEQ ID NO: 21 - DNA, 32 bp, Artificial Sequence
  Synthetic oligonucleotide primer sequence
gttatgccggacagaggaagtgacatggaggt

SEQ ID NO: 22 - DNA, 32 bp, Artificial Sequence
  Synthetic oligonucleotide primer sequence
caaggattcgcctgctgtgtggaattccattc

SEQ ID NO: 23 - DNA, 32 bp, Artificial Sequence
  Synthetic oligonucleotide primer sequence
gagtcacatggagtgagttcagcccaggagaa

SEQ ID NO: 24 - DNA, 32 bp, Artificial Sequence
  Synthetic oligonucleotide primer sequence
acaatgaccacgactgtctcttccaaccagac

SEQ ID NO: 25 - DNA, 32 bp, Artificial Sequence
  Synthetic oligonucleotide primer sequence
ttatgacttgcttctgatcttcctttctgatg

SEQ ID NO: 26 - DNA, 32 bp, Artificial Sequence
  Synthetic oligonucleotide primer sequence
tttgtaaaactagataattacactaccgactg

SEQ ID NO: 27 - DNA, 32 bp, Artificial Sequence
  Synthetic oligonucleotide primer sequence
acacagaggtgcagagaggtgacataacttcc

SEQ ID NO: 28 - DNA, 34 bp, Artificial Sequence
  Synthetic oligonucleotide primer sequence
tagcacagcccatatagtaaccactgttcaatac

SEQ ID NO: 29 - DNA, 32 bp, Artificial Sequence
  Synthetic oligonucleotide primer sequence
cttggacacccatgatgtcttggggggtggga

SEQ ID NO: 30 - DNA, 37 bp, Artificial Sequence
  Synthetic oligonucleotide primer sequence
gtgaccctccttaccttgtccttagatgcttaacatt

SEQ ID NO: 31 - DNA, 27 bp, Artificial Sequence
  Synthetic oligonucleotide primer sequence
aaccttagtccttggcacattgttgaa

SEQ ID NO: 32 - DNA, 28 bp, Artificial Sequence
  Synthetic oligonucleotide primer sequence
aacaccacattgtccatagactgatatg

SEQ ID NO: 33 - DNA, 29 bp, Artificial Sequence
  Synthetic oligonucleotide primer sequence
agtcaatgctaataatggtggcaatcacg

SEQ ID NO: 34 - DNA, 28 bp, Artificial Sequence
  Synthetic oligonucleotide primer sequence
aacaccacattgtccatagactgatatg

SEQ ID NO: 35 - DNA, 31 bp, Artificial Sequence
  Synthetic oligonucleotide primer sequence
tatgagaaggctggatcacccagaaagactg

SEQ ID NO: 36 - DNA, 31 bp, Artificial Sequence
  Synthetic oligonucleotide primer sequence
tgaggacatcatcctagtgtcgtacaagtgg

SEQ ID NO: 37 - DNA, 26 bp, Artificial Sequence
  Synthetic oligonucleotide primer sequence
tgtgttcctggattaatgcaaacagc

SEQ ID NO: 38 - DNA, 26 bp, Artificial Sequence
  Synthetic oligonucleotide primer sequence
ggacatcatcctagtgtcgtacaagt

SEQ ID NO: 39 - DNA, 26 bp, Artificial Sequence
  Synthetic oligonucleotide primer sequence
agccaatctggtgtaatggatcagac

SEQ ID NO: 40 - DNA, 26 bp, Artificial Sequence
  Synthetic oligonucleotide primer sequence
agtgctctgtaggctgctaaacttag

SEQ ID NO: 41 - DNA, 21 bp, Artificial Sequence
  Synthetic oligonucleotide primer sequence
gaaagaggcatggagagtgat

SEQ ID NO: 42 - DNA, 21 bp, Artificial Sequence
  Synthetic oligonucleotide primer sequence
ctgaagctcactgacgtctgt

SEQ ID NO: 43 - DNA, 21 bp, Artificial Sequence
  Synthetic oligonucleotide primer sequence
catggataccaagagacaact

SEQ ID NO: 44 - DNA, 20 bp, Artificial Sequence
  Synthetic oligonucleotide primer sequence
aggatcaccttccttgattc

SEQ ID NO: 45 - DNA, 20 bp, Artificial Sequence
  Synthetic oligonucleotide primer sequence
tccattaaggaaggaccttg

SEQ ID NO: 46 - DNA, 20 bp, Artificial Sequence
  Synthetic oligonucleotide primer sequence
taaacattcagcgaatgcag

SEQ ID NO: 47 - DNA, 28 bp, Artificial Sequence
  Synthetic oligonucleotide primer sequence
aaccagtctgactagtcttgcataagct

SEQ ID NO: 48 - DNA, 27 bp, Artificial Sequence
  Synthetic oligonucleotide primer sequence
gagtctgtttatgcttctgtcaggtgt

SEQ ID NO: 49 - DNA, 20 bp, Artificial Sequence
  Synthetic oligonucleotide primer sequence
ggtaaatgcacactggaacg

SEQ ID NO: 50 - DNA, 20 bp, Artificial Sequence
  Synthetic oligonucleotide primer sequence
aggattcacacctggactac

SEQ ID NO: 51 - DNA, 22 bp, Artificial Sequence
  Synthetic oligonucleotide primer sequence
atccacgtggcagcatgtgttg

SEQ ID NO: 52 - DNA, 24 bp, Artificial Sequence
  Synthetic oligonucleotide primer sequence
ctttcagaccaccagctccaggtt

SEQ ID NO: 53 - DNA, 24 bp, Artificial Sequence
  Synthetic oligonucleotide primer sequence
atgagctgaagctgctcagccttc

SEQ ID NO: 54 - DNA, 23 bp, Artificial Sequence
  Synthetic oligonucleotide primer sequence
tccaagcacaggtttgtggacag

SEQ ID NO: 55 - DNA, 27 bp, Artificial Sequence
  Synthetic oligonucleotide primer sequence
gctctgttcctggtgctttcgccgagt

SEQ ID NO: 56 - DNA, 27 bp, Artificial Sequence
  Synthetic oligonucleotide primer sequence
gacaggtctgccatccaatcgtcaggt

SEQ ID NO: 57 - DNA, 22 bp, Artificial Sequence
  Synthetic oligonucleotide primer sequence
gacactgaggtgtcaggcactt

SEQ ID NO: 58 - DNA, 21 bp, Artificial Sequence
  Synthetic oligonucleotide primer sequence
aggatcatgttcccaggctca

SEQ ID NO: 59 - DNA, 31 bp, Artificial Sequence
  Synthetic oligonucleotide primer sequence
ttgctatggctgccatgtgtcagcagcatag

SEQ ID NO: 60 - DNA, 30 bp, Artificial Sequence
  Synthetic oligonucleotide primer sequence
tctgccaccctccactcaggacacagccag

SEQ ID NO: 61 - DNA, 25 bp, Artificial Sequence
  Synthetic oligonucleotide primer sequence
ttgcagacatagggtgcacacgtgc

SEQ ID NO: 62 - DNA, 25 bp, Artificial Sequence
  Synthetic oligonucleotide primer sequence
aacaggagcgacgtcgctaagctag

SEQ ID NO: 63 - DNA, 23 bp, Artificial Sequence
  Synthetic oligonucleotide primer sequence
agcacatcagtgattaagtcagg

SEQ ID NO: 64 - DNA, 23 bp, Artificial Sequence
  Synthetic oligonucleotide primer sequence
gattcgatggacaacatgctcct

SEQ ID NO: 65 - DNA, 23 bp, Artificial Sequence
  Synthetic oligonucleotide primer sequence
ttgggaatctctgaatgacagtg

SEQ ID NO: 66 - DNA, 22 bp, Artificial Sequence
  Synthetic oligonucleotide primer sequence
ggtttggaagcctaggcaggaa

SEQ ID NO: 67 - DNA, 22 bp, Artificial Sequence
  Synthetic oligonucleotide primer sequence
gagaggccttggctctctctga

SEQ ID NO: 68 - DNA, 22 bp, Artificial Sequence
  Synthetic oligonucleotide primer sequence
tctctaacaccatgcagagtgg

SEQ ID NO: 69 - DNA, 23 bp, Artificial Sequence
  Synthetic oligonucleotide primer sequence
ctgatgtccagattcctgctagt

SEQ ID NO: 70 - DNA, 25 bp, Artificial Sequence
  Synthetic oligonucleotide primer sequence
acctccagcatttattcatgccatg

SEQ ID NO: 71 - DNA, 34 bp, Artificial Sequence
  Synthetic oligonucleotide primer sequence
agaaggaaatctaggcttagagactccacctccc

SEQ ID NO: 72 - DNA, 34 bp, Artificial Sequence
  Synthetic oligonucleotide primer sequence
gcatatgattccacttatatgaggtacctagaat

SEQ ID NO: 73 - DNA, 22 bp, Artificial Sequence
  Synthetic oligonucleotide primer sequence
tggatgtggtggaactaggtgg

SEQ ID NO: 74 - DNA, 22 bp, Artificial Sequence
  Synthetic oligonucleotide primer sequence
aaccgatccctgaccggttctg

SEQ ID NO: 75 - DNA, 26 bp, Artificial Sequence
  Synthetic oligonucleotide primer sequence
agaggtggtaactttggaagtcctgg

SEQ ID NO: 76 - DNA, 28 bp, Artificial Sequence
  Synthetic oligonucleotide primer sequence
ggtatgtgcactcctcagagcaggcata

SEQ ID NO: 77 - DNA, 28 bp, Artificial Sequence
  Synthetic oligonucleotide primer sequence
tggtcagatggatagatggcatcacctc

SEQ ID NO: 78 - DNA, 29 bp, Artificial Sequence
  Synthetic oligonucleotide primer sequence
atcacatcttgctgatgaggaaatgcagg

SEQ ID NO: 79 - DNA, 30 bp, Artificial Sequence
  Synthetic oligonucleotide primer sequence
tcacagtctggtggcatagtacctaaattg

SEQ ID NO: 80 - DNA, 28 bp, Artificial Sequence
  Synthetic oligonucleotide primer sequence
ctcccaggttgtagatgatctcaaacac

SEQ ID NO: 81 - DNA, 20 bp, Artificial Sequence
  Synthetic oligonucleotide primer sequence
tgcagctcctgatctaggat

SEQ ID NO: 82 - DNA, 19 bp, Artificial Sequence
  Synthetic oligonucleotide primer sequence
agagcaggcataactgcag

SEQ ID NO: 83 - DNA, 22 bp, Artificial Sequence
  Synthetic oligonucleotide primer sequence
attagagatctcagacagggtg

SEQ ID NO: 84 - DNA, 22 bp, Artificial Sequence
  Synthetic oligonucleotide primer sequence
aactgggcatgactttgatagg

SEQ ID NO: 85 - DNA, 26 bp, Artificial Sequence
  Synthetic oligonucleotide primer sequence
acctcagtcactcttgggaatctctg

SEQ ID NO: 86 - DNA, 26 bp, Artificial Sequence
  Synthetic oligonucleotide primer sequence
tagaagtgtattccctctcagctgtg

SEQ ID NO: 87 - DNA, 22 bp, Artificial Sequence
  Synthetic oligonucleotide primer sequence
tgcagggtatcgaggaggtggc

SEQ ID NO: 88 - DNA, 22 bp, Artificial Sequence
  Synthetic oligonucleotide primer sequence
tgcaatatctccaagggatgcc

SEQ ID NO: 89 - DNA, 32 bp, Artificial Sequence
  Synthetic oligonucleotide primer sequence
ggccccttaagtattcacacattacagaaata

SEQ ID NO: 90 - DNA, 32 bp, Artificial Sequence
  Synthetic oligonucleotide primer sequence
gttgaaacttgatctcccagtgttggcagtgg

SEQ ID NO: 91 - DNA, 34 bp, Artificial Sequence
  Synthetic oligonucleotide primer sequence
cgaggtggaaggagtctgggaggcccgctcacaa

SEQ ID NO: 92 - DNA, 34 bp, Artificial Sequence
  Synthetic oligonucleotide primer sequence
agacacataatagaggctcaacatgcaagcttcc

SEQ ID NO: 93 - DNA, 33 bp, Artificial Sequence
  Synthetic oligonucleotide primer sequence
ggccatgcactccaactgccaactgctgagtct

SEQ ID NO: 94 - DNA, 33 bp, Artificial Sequence
  Synthetic oligonucleotide primer sequence
tcacctcccagcctgatgtccagcacttcctcc

SEQ ID NO: 95 - DNA, 20 bp, Artificial Sequence
  Synthetic oligonucleotide primer sequence
tggtagcacataagcctctg

SEQ ID NO: 96 - DNA, 20 bp, Artificial Sequence
  Synthetic oligonucleotide primer sequence
atcacaatggccagtcagtc

SEQ ID NO: 97 - DNA, 20 bp, Artificial Sequence
  Synthetic oligonucleotide primer sequence
tcctaacatggaactcatgg

SEQ ID NO: 98 - DNA, 20 bp, Artificial Sequence
  Synthetic oligonucleotide primer sequence
ttaccacctccttcaggaag

SEQ ID NO: 99 - DNA, 20 bp, Artificial Sequence
  Synthetic oligonucleotide primer sequence
ccaacatctctgagcaccat

SEQ ID NO: 100 - DNA, 19 bp, Artificial Sequence
  Synthetic oligonucleotide primer sequence
tgagtgtctgagatcaggc

SEQ ID NO: 101 - DNA, 22 bp, Artificial Sequence
  Synthetic oligonucleotide primer sequence
acaaacaaccatccacagtggg

SEQ ID NO: 102 - DNA, 23 bp, Artificial Sequence
  Synthetic oligonucleotide primer sequence
tctgagaaaggcaggagatctag

SEQ ID NO: 103 - DNA, 24 bp, Artificial Sequence
  Synthetic oligonucleotide primer sequence
aaagacaagtcaggctttgagcac

SEQ ID NO: 104 - DNA, 23 bp, Artificial Sequence
  Synthetic oligonucleotide primer sequence
tatgaagtccaatactgaacatg

SEQ ID NO: 105 - DNA, 24 bp, Artificial Sequence
  Synthetic oligonucleotide primer sequence
tgtggtagtgcatgcctgtaatcc

SEQ ID NO: 106 - DNA, 24 bp, Artificial Sequence
  Synthetic oligonucleotide primer sequence
atggctgtgtgtactaacagtcgc

SEQ ID NO: 107 - DNA, 26 bp, Artificial Sequence
  Synthetic oligonucleotide primer sequence
agctccagaggacctcagactctatc

SEQ ID NO: 108 - DNA, 26 bp, Artificial Sequence
  Synthetic oligonucleotide primer sequence
tgaggtatgactcctcaggtagacag

SEQ ID NO: 109 - DNA, 24 bp, Artificial Sequence
  Synthetic oligonucleotide primer sequence
cctgcttccatggatatccaggct

SEQ ID NO: 110 - DNA, 26 bp, Artificial Sequence
  Synthetic oligonucleotide primer sequence
ctcagtctgtaggagacaggaggtga

SEQ ID NO: 111 - DNA, 25 bp, Artificial Sequence
  Synthetic oligonucleotide primer sequence
ctgtggagatcgtagacacctccaa

SEQ ID NO: 112 - DNA, 25 bp, Artificial Sequence
  Synthetic oligonucleotide primer sequence
actagaggtggctcctgtccttgtc

SEQ ID NO: 113 - DNA, 26 bp, Artificial Sequence
  Synthetic oligonucleotide primer sequence
taactacacgctgctggatgagcatc

SEQ ID NO: 114 - DNA, 27 bp, Artificial Sequence
  Synthetic oligonucleotide primer sequence
agaccagctttgtgtgttccagggaag

SEQ ID NO: 115 - DNA, 25 bp, Artificial Sequence
  Synthetic oligonucleotide primer sequence
ctgtggagatcgtagacacctccaa

SEQ ID NO: 116 - DNA, 26 bp, Artificial Sequence
  Synthetic oligonucleotide primer sequence
ctctgtagattcttcctcatctgccc

SEQ ID NO: 117 - DNA, 26 bp, Artificial Sequence
  Synthetic oligonucleotide primer sequence
taactacacgctgctggatgagcatc

SEQ ID NO: 118 - DNA, 23 bp, Artificial Sequence
  Synthetic oligonucleotide primer sequence
tgatcaacagggaggaggcattt

SEQ ID NO: 119 - DNA, 20 bp, Artificial Sequence
  Synthetic oligonucleotide primer sequence
ctgccccctccagcacctta

SEQ ID NO: 120 - DNA, 18 bp, Artificial Sequence
  Synthetic oligonucleotide primer sequence
cctagcgagagctcccag

SEQ ID NO: 121 - DNA, 18 bp, Artificial Sequence
  Synthetic oligonucleotide primer sequence
gacagctcgggccatgac

SEQ ID NO: 122 - DNA, 28 bp, Artificial Sequence
  Synthetic oligonucleotide primer sequence
tgggcagatgaggaagaatctacagagc

SEQ ID NO: 123 - DNA, 28 bp, Artificial Sequence
  Synthetic oligonucleotide primer sequence
ttaccacagcgccatgagttgttgtaag

SEQ ID NO: 124 - DNA, 28 bp, Artificial Sequence
  Synthetic oligonucleotide primer sequence
acatgaggtcctcctacctctagtccag

SEQ ID NO: 125 - DNA, 34 bp, Artificial Sequence
  Synthetic oligonucleotide primer sequence
ctgtggagatcgtagacacctccaaccctgagct

SEQ ID NO: 126 - DNA, 34 bp, Artificial Sequence
  Synthetic oligonucleotide primer sequence
cagatagcctctctacctcactgggatttggaca

SEQ ID NO: 127 - DNA, 29 bp, Artificial Sequence
  Synthetic oligonucleotide primer sequence
taaggaccaaacgagatcacaggtgtgga

SEQ ID NO: 128 - DNA, 29 bp, Artificial Sequence
  Synthetic oligonucleotide primer sequence
agcctctctacctcactgggatttggaca

SEQ ID NO: 129 - DNA, 30 bp, Artificial Sequence
  Synthetic oligonucleotide primer sequence
cgagtcactagaagtaggatcttggtttgt

SEQ ID NO: 130 - DNA, 30 bp, Artificial Sequence
  Synthetic oligonucleotide primer sequence
ggtttgttctacctcactgggatttggaca

SEQ ID NO: 131 - DNA, 28 bp, Artificial Sequence
  Synthetic oligonucleotide primer sequence
gtagacaggtgatggcatagaggcttct

SEQ ID NO: 132 - DNA, 28 bp, Artificial Sequence
  Synthetic oligonucleotide primer sequence
tggtactgaatctgccagcctagagaac

SEQ ID NO: 133 - DNA, 28 bp, Artificial Sequence
  Synthetic oligonucleotide primer sequence
aggcacttcccagagaagcagagaattg

SEQ ID NO: 134 - DNA, 29 bp, Artificial Sequence
  Synthetic oligonucleotide primer sequence
tgtggctgaatctctttaaagaggtcagg

SEQ ID NO: 135 - DNA, 25 bp, Artificial Sequence
  Synthetic oligonucleotide primer sequence
tcagagaatttgcatcagggttctc

SEQ ID NO: 136 - DNA, 23 bp, Artificial Sequence
  Synthetic oligonucleotide primer sequence
taagcaaccatctgtcacagacc

SEQ ID NO: 137 - DNA, 25 bp, Artificial Sequence
  Synthetic oligonucleotide primer sequence
tggaaccattgtaagttgaggactt

SEQ ID NO: 138 - DNA, 25 bp, Artificial Sequence
  Synthetic oligonucleotide primer sequence
gagatgaggtctcacgtctcaaact

SEQ ID NO: 139 - DNA, 29 bp, Artificial Sequence
  Synthetic oligonucleotide primer sequence
atcaactgggagtttcaggtttatcagcc

SEQ ID NO: 140 - DNA, 28 bp, Artificial Sequence
  Synthetic oligonucleotide primer sequence
aaggcaaattgtcctgctaagctcggtg

SEQ ID NO: 141 - DNA, 30 bp, Artificial Sequence
  Synthetic oligonucleotide primer sequence
aatgagaccatgtgctacaagtacgaagtg

SEQ ID NO: 142 - DNA, 30 bp, Artificial Sequence
  Synthetic oligonucleotide primer sequence
tttgttcactcttacctaggtgagagcctg

SEQ ID NO: 143 - DNA, 29 bp, Artificial Sequence
  Synthetic oligonucleotide primer sequence
gatcgtccacaaggttgactacgaccagt

SEQ ID NO: 144 - DNA, 29 bp, Artificial Sequence
  Synthetic oligonucleotide primer sequence
tcattgattctcacctcacagatctaagc

SEQ ID NO: 145 - DNA, 23 bp, Artificial Sequence
  Synthetic oligonucleotide primer sequence
taggataaggtgtactgctactt

SEQ ID NO: 146 - DNA, 25 bp, Artificial Sequence
  Synthetic oligonucleotide primer sequence
gaagacaaatccttgtgtttaacca

SEQ ID NO: 147 - DNA, 23 bp, Artificial Sequence
  Synthetic oligonucleotide primer sequence
aacacatggagatatcactgagc

SEQ ID NO: 148 - DNA, 25 bp, Artificial Sequence
  Synthetic oligonucleotide primer sequence
cctaaatccaatgacaagtgtcctt

SEQ ID NO: 149 - DNA, 28 bp, Artificial Sequence
  Synthetic oligonucleotide primer sequence
cttaagtcctacagtgtccatggagata

SEQ ID NO: 150 - DNA, 28 bp, Artificial Sequence
  Synthetic oligonucleotide primer sequence
catcagtgatgtgttaaaggttatattc

SEQ ID NO: 151 - DNA, 25 bp, Artificial Sequence
  Synthetic oligonucleotide primer sequence
tcactgatatgtgctttacttctgg

SEQ ID NO: 152 - DNA, 26 bp, Artificial Sequence
  Synthetic oligonucleotide primer sequence
aggatttcctggcaaatgcagtcttc

SEQ ID NO: 153 - DNA, 27 bp, Artificial Sequence
  Synthetic oligonucleotide primer sequence
gtcttgtacctaatgagcaaattatct

SEQ ID NO: 154 - DNA, 27 bp, Artificial Sequence
  Synthetic oligonucleotide primer sequence
gcattgtatggatattcaactcaaatt

SEQ ID NO: 155 - DNA, 19 bp, Artificial Sequence
  Synthetic oligonucleotide primer sequence
gaattagtgccttggtaga

SEQ ID NO: 156 - DNA, 20 bp, Artificial Sequence
  Synthetic oligonucleotide primer sequence
gtattgggaattagtgcctt

SEQ ID NO: 157 - DNA, 22 bp, Artificial Sequence
  Synthetic oligonucleotide primer sequence
cagaagttattgctttgcaact

SEQ ID NO: 158 - DNA, 30 bp, Artificial Sequence
  Synthetic oligonucleotide primer sequence
ctctacaatgctattggtaggtgtaactta

SEQ ID NO: 159 - DNA, 30 bp, Artificial Sequence
  Synthetic oligonucleotide primer sequence
cacaacagcatttatcctcaatgtcaaaga

SEQ ID NO: 160 - DNA, 32 bp, Artificial Sequence
  Synthetic oligonucleotide primer sequence
agcagttagcaatgattcttcaccaacttgtg

SEQ ID NO: 161 - DNA, 32 bp, Artificial Sequence
  Synthetic oligonucleotide primer sequence
cctggagtcacgctacaactaattacatttct

SEQ ID NO: 162 - DNA, 24 bp, Artificial Sequence
  Synthetic oligonucleotide primer sequence
ttcctagagccatacagatacttg

SEQ ID NO: 163 - DNA, 23 bp, Artificial Sequence
  Synthetic oligonucleotide primer sequence
gctgaatggaaacggatgctatt